SEQUENCE LISTING (2 sequences)

SEQ ID NO: 1 - protein, 13 aa, synthetic construct
AAVRPLWVRMEAA

SEQ ID NO: 2 - protein, 21 aa, synthetic construct
MEVGWYRSPFSRVVHLYRNGK

(19) United States
(12) Patent Application Publication
(10) Pub. No.: US 2026/0014271 A1
(43) Pub. Date: Jan. 15, 2026

(54) METABOLIC TAGGING AND TARGETING OF RED BLOOD CELLS

(71) Applicant: The Board of Trustees of the University of Illinois, Urbana, IL (US)

(72) Inventors: Hua Wang, Urbana, IL (US); Yusheng Liu, Urbana, IL (US)

(73) Assignee: The Board of Trustees of the University of Illinois, Urbana, IL (US)

(21) Appl. No.: 19/262,448

(22) Filed: Jul. 8, 2025

Related U.S. Application Data
(60) Provisional application No. 63/669,055, filed on Jul. 9, 2024.

(51) Int. Cl. A61K 47/69; A61K 47/54; A61K 49/18; A61P 3/10

(52) U.S. Cl. CPC A61K 47/6901; A61K 47/549; A61K 49/1896; A61P 3/10

(57) ABSTRACT

Modified red blood cells (RBCs) including one or more surface proteins or lipids covalently linked to a cargo by click chemistry are provided. In some examples, the cargo is a cancer therapeutic agent, an autoimmune antigen, or an antigen of a bacterial or viral agent. Also provided are methods of treating a subject with a disease or disorder with the modified RBCs or methods of treating a subject with a disease or disorder or performing imaging analysis of a subject, including administering to the subject a composition including an azido-modified sugar moiety, thereby generating red blood cells comprising one or more azido-labeled surface proteins and administering to the subject a composition including a cargo for treating or inhibiting the disease or disorder, wherein the cargo is capable of covalently binding to the one or more azido-labeled surface proteins or lipids.

### CROSS REFERENCE TO RELATED APPLICATIONS

This application claims priority to U.S. Provisional Application No. 63/669,055, filed Jul. 9, 2024, which is incorporated by reference in its entirety.

### ACKNOWLEDGMENT OF GOVERNMENT SUPPORT

This invention was made with government support under R01 CA274738 and R21 CA270872 awarded by the National Institute of Health and under 2143673 awarded by the National Science Foundation. The government has certain rights in the invention.

### FIELD

This disclosure relates to modified red blood cells including one or more proteins or lipids covalently linked to a cargo, and methods of their use, for example for treatment or inhibition of disease.

### INCORPORATION OF ELECTRONIC SEQUENCE LISTING

The Sequence Listing is submitted as an XML file in the form of the file named “7950-108802-03_Sequence_Listing.xml” (2,815 bytes), which was created on Jun. 28, 2025, which is incorporated by reference herein.

### BACKGROUND

RBCs constitute 40-45% of whole blood volume and >99% of blood cells, and are responsible for transporting oxygen to all the tissues in the body. Due to their long life span (˜120 days in human and ˜45 days in mice) and ability to access deep tissues, RBCs are an attractive engineering target for drug delivery, imaging, hemostasis, vaccination, and other applications. For example, drugs bonded or conjugated onto RBCs can exhibit significantly prolonged blood circulation. Compared to hydrophilic polymers (e.g., polyethylene glycol), nanoparticles, microparticles, and other drug delivery vehicles, RBCs can improve the blood retention and tissue accumulation of drugs with minimal immunogenicity, potentially resulting in enhanced efficacy of drugs for treating cancers, injured tissues, infectious diseases, and autoimmune disorders. In addition to long lifespan and excellent tissue accessibility, RBCs that become aged or damaged or express certain ‘removal’ immune markers are cleared by phagocytic cells (e.g., macrophages) at a rate of millions per second in a non-inflammatory manner.

Taking advantage of this phenomenon, RBCs conjugated with autoantigens have previously been adoptively transferred to induce antigen-specific tolerance against autoimmune disorders (Cremel et al., Int. J. Pharm. 443:39-49, 2013; Pishesha et al., Proc. Natl. Acad. Sci. 114:3157-3162, 2017; Kontos et al., Proc. Natl. Acad. Sci. USA 110: E60-E68, 2013). These and other emerging applications have demonstrated the promise of engineered RBCs to innovate or improve therapies for treating different diseases. However, facile yet safe and effective methods for in vivo surface engineering of RBCs still do not exist to date, often necessitating the adoptive transfer of ex vivo engineered RBCs.

In vivo surface engineering and cargo attachment of RBCs remains a significant challenge. RBCs can be modified via non-covalent or covalent methods. Non-covalent methods involve physical adsorption of cargos onto RBC membranes via electrostatic interactions or binding of a targeting ligand to the cell-surface receptors (e.g., Ter119). The former is limited by the weak interactions between cargos and RBCs and the need of custom-designed adsorbable structures, while the latter is limited by low abundance of RBC receptors and unknown biological impact of receptor occupation. Methods that enable covalent conjugation of cargos to the membrane of RBCs have also previously been developed. For example, cell-surface amine groups were utilized to covalently conjugate carboxyl-bearing molecules. However, the number of amine groups per RBC is low and the modified RBCs exhibited reduced survival and likely impaired functions (Muzykantov, Exp. Opin. Drug Deliv. 7:403-427, 2010; Rossi et al., Biomaterials 31:4167-4178, 2010). Another covalent method involves installation of a peptide motif to the end of cell-surface proteins via gene transduction, followed by conjugation with (Gly)ₙ-bearing molecules in the presence of sortase. A notable drawback of this strategy is the necessity of introducing a sortase-cleavable motif via gene transduction. Furthermore, all of these methods are difficult to apply in vivo because of the poor specificity, low efficiency, and potential toxicity.

### SUMMARY

Direct engineering of circulating RBCs in vivo remains a significant challenge. Provided herein are novel strategies to enable efficient conjugation of cargos to RBCs, both in vitro and in vivo.

Provided herein are modified red blood cells that include one or more surface proteins or lipids covalently linked to a cargo by click chemistry. In some examples, the click chemistry is azide-alkyne click chemistry, tetrazine-norbornene click chemistry, tetrazine-cyclooctene click chemistry, or maleimide-thiol click chemistry. In particular examples, the red blood cells are human red blood cells. In some examples, the cargo is an autoantigen, a compound for treatment of cancer, insulin, an antigen (such as a viral or bacterial antigen), and antibiotic, an antiviral agent, or an antibody or fragment thereof.

In some examples, the click chemistry includes an azido-labeled sugar moiety, for example, tetra-acetylated N-azidoacetyl-D-mannosamine (Ac₄ManNAz, also referred to as AAM), N-azidoacetyl-D-mannosamine (ManNAz), tetra-acetylated N-azidoacetyl-D-galactosamine (Ac₄GalNAz), N-azidoacetyl-D-galactosamine (GalNAz), tetra-acetylated N-azidoacetyl-D-glucosamine (Ac₄GlcNAz), N-azidoacetyl-D-glucosamine (GlcNAz), or N-Acetyl-9-azido-9-deoxy-neuraminic acid (9-AzNeu5Ac). In one example, the azido-labeled sugar moiety is Ac₄ManNAz.

Also provided are compositions including a disclosed modified red blood cell and a pharmaceutically acceptable carrier.

Further provided are methods for treating or inhibiting a disease or disorder. In some examples, the method is an in vivo method, including administering to a subject a composition including an azido-modified sugar moiety, thereby generating red blood cells comprising one or more azido-labeled surface proteins and administering to the subject a composition including a cargo for treating or inhibiting the disease, wherein the cargo is capable of covalently binding to the one or more azido-labeled surface proteins or lipids. In some examples, the cargo is administered to the subject after the composition including the azido-modified sugar moiety.

Also provided are ex vivo methods for treating or inhibiting a disease or disorder. In some examples, the methods include contacting a preparation of red blood cells obtained from a subject with the disease or disorder with a composition including an azido-modified sugar for a sufficient period of time for the red blood cells to express one or more azido-labeled surface proteins or lipids (for example, for about 1-120 hours) and contacting the red blood cells expressing one or more azido-labeled surface proteins or lipids with a composition including a cargo for treating or inhibiting the disease, wherein the cargo is capable of covalently binding to the one or more azido-labeled surface proteins or lipids, thereby producing a population of modified red blood cells. The subject is then administered the population of modified red blood cells.

In some methods of treating or inhibiting a disease or disorder, the subject may have cancer (such as breast cancer or glioblastoma) or metastatic cancer and be administered a cargo including a cancer therapeutic agent. In other examples of methods of treating or inhibiting a disease or disorder, the subject may have an autoimmune disease and be administered a cargo including an autoantigen. In further examples methods of treating or inhibiting a disease or disorder, the subject may have or be at risk of an infectious disease (such as a bacterial or viral disease) and be administered a cargo including an antigen of the bacterial or viral agent causing the disease. In further examples, the subject may have or be at risk of an infectious disease (such as a bacterial or viral disease) and be administered a cargo including an antibiotic or an antiviral agent.

In some examples, the composition including the azido-modified sugar administered to the subject, or contacted with the RBCs, is tetra-acetylated N-azidoacetyl-D-mannosamine (Ac4ManNAz), N-azidoacetyl-D-mannosamine (ManNAz), tetra-acetylated N-azidoacetyl-D-galactosamine (Ac4GalNAz), N-azidoacetyl-D-galactosamine (GalNAz), tetra-acetylated N-azidoacetyl-D-glucosamine (Ac4GlcNAz), N-azidoacetyl-D-glucosamine (GlcNAz), or N-Acetyl-9-azido-9-deoxy-neuraminic acid (9-AzNeu5Ac), for example, Ac4ManNAz. In additional examples, the cargo is capable of covalently binding to the one or more azido-labeled surface proteins or lipids is capable of covalent binding by click chemistry, such as azide-alkyne click chemistry. In additional examples, the cargo capable of covalently binding to the one or more azido-labeled surface proteins or lipids includes dibenzocyclooctyne.

In further examples, methods of in vivo imaging and provided, the methods including administering to a subject a composition including an azido-modified sugar moiety, thereby generating red blood cells including one or more azido-labeled surface proteins; administering to the subject a composition including an imaging agent, wherein the imaging agent is capable of covalently binding to the one or more azido-labeled surface proteins or lipids; and performing an imaging analysis of the subject one or more times.

The foregoing and other features of this disclosure will become more apparent from the following detailed description, which proceeds with reference to the accompanying figures.

### BRIEF DESCRIPTION OF THE DRAWINGS

FIGS. 1A-1D illustrate an exemplary in vivo RBC labeling and targeting method. FIG. 1A is a schematic diagram of in vivo metabolic glycan labeling of RBCs with azido groups for subsequent conjugation of DBCO-cargo via click chemistry. Azido tags on nucleus-free RBCs can persist for >42 days (nearly life-span of mouse RBCs) while azido tags on DNA-containing cells become undetectable after 3 days, enabling specific conjugation of DBCO-cargos to circulating RBCs at 3 days or later. FIG. 1B is a schematic diagram showing in vivo conjugation of fluorophores to RBCs for fluorescence imaging of blood vessels and tumors. FIG. 1C shows in vivo conjugation of DBCO-DOTA-Gd to RBCs for long-term magnetic resonance imaging (MRI) of brain vasculatures. FIG. 1D is a schematic showing in vivo conjugation of drugs to RBCs for improved pharmacokinetics and therapeutic efficacy.

FIGS. 2A-2O illustrate that systemically administered azido-sugar can metabolically label RBCs with azido groups in vivo, and azido groups persist on circulating RBCs for >42 days in mice. FIG. 2A is a schematic illustration of in vivo metabolic labeling of circulating RBCs. For FIGS. 2B-2K, AAM was intravenously injected into C57BL/6 mice twice daily for three days. Isolated RBCs from mice were incubated with DBCO-Cy5 and nucleic acid stain (SYTO) for 1 h. FIG. 2B is a representative flow cytometry plot of RBCs at 14 days post AAM injections. FIG. 2C shows fluorescence images and FIG. 2D shows confocal images of RBCs harvested at 14 days post AAM injections. Scale bar represents 10 μm. Also shown are the percentage of RBCs positive for Cy5 fluorescence (% Cy5⁺ RBCs) (FIG. 2E); the mean Cy5 fluorescence intensity of all RBCs (FIG. 2F); and the Cy5 fluorescence intensity ratio of RBCs in AAM-treated versus PBS-treated mice (FIG. 2G) harvested at different times post AAM injections and stained with DBCO-Cy5. Also shown are % Cy5⁺ cells among DNA⁺ cells (FIG. 2H) and mean Cy5 fluorescence intensity of DNA⁺ cells in the blood (FIGS. 2I) at 1, 3, and 5 days post AAM injections. FIG. 2J shows the number of Cy5⁺ white blood cells (WBCs) and RBCs in the blood at day 0.5, 2.5, 4.5, and 7.5 post AAM injections. FIG. 2K shows corresponding normalized percentages of Cy5⁺ WBCs and RBCs at those time points. FIG. 2L is a schematic illustration of purification of azido-labeled RBCs via DBCO-desthiobiotin conjugation and streptavidin-microbead pull-down. FIG. 2M shows representative PE-streptavidin histograms of RBCs before and after purification. FIG. 2N shows fluorescence images of RBCs before and after purification. Scale bar represents 20 μm. FIG. 2O shows Western blot analysis of proteins extracted from azido⁺ and azido⁻ RBCs, with enriched bands in azido⁺ RBCs indicated by boxes. All numerical data are presented as mean±SD (one-way ANOVA with post hoc Fisher's LSD test was used; 0.01<*P≤0.05; **P≤0.01; ***P≤0.001; ****P≤0.0001).

FIGS. 3A-3H illustrate that azido-sugars can metabolically label surface glycoproteins and glycolipids on RBCs in the blood and precursor cells in the bone marrow. FIG. 3A shows a schematic illustration of the isolation of membrane and cytoplasmic protein fractions from RBCs collected 7 days after AAM or PBS injection. RBCs were incubated with DBCO-desthiobiotin prior to protein fractionation and western blot analyses. FIGS. 3B-3C show Western blot analysis of desthiobiotin-tagged proteins. FIG. 3B compares cytoplasmic, membrane, and supernatant fractions, with desthiobiotin signal detected using streptavidin-HRP. FIG. 3C shows membrane protein samples with or without PNGase F digestion to confirm glycoprotein identity. FIG. 3D shows a representative HPLC profile of lipids from RBCs at 7 days post injections of AAM or PBS. RBCs isolated from AAM- or PBS-treated mice were incubated with DBCO-Cy5, followed by lipid extraction and HPLC analysis (λₑₓ: 645 nm, λₑₘ: 660 nm). 4T1 cells treated with AAM for 24 h in vitro were used as controls. For FIGS. 3E-3F, bone marrow cells were collected at 48 h post injections of AAM or PBS, incubated with DBCO-Cy5, and stained for erythroid lineage markers prior to flow cytometry analysis. FIG. 3E shows the mean Cy5 fluorescence intensity of different RBC precursor populations. FIG. 3F shows the Cy5 fluorescence intensity ratio (AAM/PBS) for different RBC precursor cells. Pro: proerythroblast; Baso: basophilic erythroblast; Poly: polychromatophilic erythroblast; Ortho: orthochromatophilic erythroblast; Retic: reticulocyte. FIGS. 3G-3H show RBC labeling following intravenous injection of AAM, AAGal, AAGlu, or PBS into C57BL/6 mice twice daily for two days. RBCs were collected at multiple time points and stained with DBCO-Cy5. FIG. 3G shows the percentage of Cy5⁺ RBCs at 1, 2, 5, 7, and 14 days post injection. FIG. 3H shows the corresponding mean Cy5 fluorescence intensity of RBCs at the same time points. All the numerical data are presented as mean±SD (one-way ANOVA with post hoc Fisher's LSD test was used; 0.01<*P≤0.05; **P≤0.01; ***P≤0.001; ****P≤0.0001).

FIGS. 4A-4I illustrates that in vivo metabolic labeling of RBCs does not induce noticeable toxicity to RBCs, WBCs and healthy tissues. FIG. 4A shows representative microscopic images of RBCs harvested from mice at 1 or 3 days post injections of AAM or PBS. FIG. 4B shows % singlet RBCs harvested at 1 or 3 days post the last injection of AAM or PBS. FIG. 4C shows intracellular ATP levels of RBCs collected from mice at 1 or 3 days post the last injection of AAM or PBS. FIG. 4D shows intracellular NAD(P)H levels of RBCs collected from mice at 1 or 3 days post the last injection of AAM or PBS. Counts of RBCs (FIG. 4E) and WBCs (FIG. 4F) at different times post injections of AAM or PBS are shown. FIG. 4G represents the % of different types of WBCs at different time points post injections of AAM or PBS. FIG. 4H shows representative images of H&E stained tissue sections at 6 weeks post injections of AAM or PBS. Scale bar represents 100 μm. FIG. 4I shows Western blot analysis of proteins extracted from different organs at 4 days post AAM or PBS injection. Proteins were extracted, and incubated with DBCO-biotin and analyzed by SDS-PAGE. Biotin signal was detected using streptavidin-HRP. FIG. 4J shows confocal images of tissue sections collected at 4 days post AAM or PBS injection, stained with DBCO-Cy5 and DAPI. Scale bar represents 50 μm. All the numerical data are presented as mean±SD (one-way ANOVA with post hoc Fisher's LSD test was used; 0.01<*P≤0.05; **P≤0.01; ***P≤0.001; ****P≤0.0001).

FIGS. 5A-5J illustrate that DBCO-molecules administered at 3 days post AAM injections can target azido-labeled RBCs in vivo and persist on RBC membranes for >35 days. FIG. 5A is a schematic diagram of the in vivo RBC labeling and targeting study. AAM or PBS was intravenously injected into C57BL/6 mice twice daily for three days, followed by intravenous injection of DBCO-Cy5 at three days post the last AAM injection. FIG. 5B shows % Cy5⁺ RBCs harvested from AAM- or PBS-treated mice at different times post injection of DBCO-Cy5. FIG. 5C shows mean Cy5 fluorescence intensity of RBCs harvested from AAM- or PBS-treated mice at different times post injection of DBCO-Cy5. FIG. 5D shows Cy5 fluorescence intensity ratio (AAM/PBS) of RBCs harvested at different times post DBCO-Cy5 injection. FIG. 5E shows IVIS imaging of blood at 1, 4, 7, and 14 days post injection of DBCO-Cy5. FIG. 5F shows Cy5 fluorescence intensity of blood in FIG. 5E. Also shown are mean Cy5 fluorescence intensity of DNA⁺ cells (FIG. 5G) and Cy5 fluorescence intensity ratio (AAM/PBS) of DNA⁺ cells in the blood (FIG. 5H) at different times post DBCO-Cy5 injection. For FIGS. 5I and 5J, AAM or PBS was intravenously injected into C57BL/6 mice twice daily for three days, followed by intravenous injection of DBCO-Cy5 at 4 days after the last AAM injection. FIG. 5I shows confocal images of sectioned tissues collected 24 h after DBCO-Cy5 injection. Tissue sections were stained with DAPI. Scale bar represents 50 μm. FIG. 5J shows Cy5 fluorescence intensity measurements of the tissues shown in FIG. 5I. All the numerical data are presented as mean±SD (one-way ANOVA with post hoc Fisher's LSD test was used; 0.01<*P≤0.05; **P≤0.01; ***P≤0.001; ****P≤0.0001).

FIGS. 6A-6L illustrate that in vivo conjugation of fluorophore and Gd agent to RBCs enables long-term fluorescence imaging and MRI of blood vessels and tissues. For FIGS. 6A-6F, AAM or PBS was i.v. injected into Balb/c mice twice daily for three days, followed by subcutaneous injection of 4T1 tumor cells on day −3 and i.p. injection of DBCO-Cy5 on day 0. FIG. 6A is a schematic diagram of the imaging study. FIG. 6B shows Cy5 fluorescence intensity of RBCs at different time points post injection of DBCO-Cy5. FIG. 6C shows IVIS imaging of Balb/c mice at 21 days post injection of DBCO-Cy5. FIG. 6D shows Cy5 fluorescence intensity of tumors in FIG. 6C. FIG. 6E shows ex vivo imaging of 4T1 tumors harvested at 21 days post injection of DBCO-Cy5. FIG. 6F shows Cy5 fluorescence intensity of 4T1 tumors in FIG. 6E. For FIGS. 6G-6L, AAM or PBS was intravenously injected into C57BL/6 mice twice daily for three days, followed by intravenous injection of DBCO-DOTA-Gd at four days post the last injection of AAM or PBS. T1-weighted CE-MRI scan of mouse brain was performed before and at different times after the injection of DBCO-DOTA-Gd. FIG. 6G is a schematic diagram of the MRI study. AAM or PBS was intravenously injected into C57BL/6 mice twice daily for three days, followed by intravenous injection of DBCO-DOTA-Gd at four days post the last injection of AAM or PBS. T1-weighted CE-MRI scan of mouse brain was performed before and at different times after the injection of DBCO-DOTA-Gd. FIG. 6H shows coronal view of 3D maximum intensity projection images of AAM- or PBS-treated mice at different times. Blood vessels are indicated by arrows. FIGS. 6I and 6K show coronal view comparisons of AAM- and PBS-treated mice at 4 or 11 days post injection of DBCO-DOTA-Gd. The blood vessels of interest are circled. FIGS. 6J and 6L show contrast enhancement analysis of coronal view 1 (FIG. 6I) and view 2 (FIG. 6K), respectively. The signal from blood vessels and the background were quantified by ImageJ. Three visible vessels were analyzed for FIGS. 6I and 6J, and two visible vessels for FIGS. 6K and 6L. All of the numerical data are presented as mean±SD (two-tailed Welch's t-test was used; 0.01<*P≤0.05; **P≤0.01; ***P≤0.001; ****P≤0.0001).

FIGS. 7A-7H illustrate in vivo conjugation of drugs to RBCs improves the pharmacokinetics and therapeutic efficacy. FIG. 7A is a schematic illustration for the conjugation of DBCO-insulin to azido-labeled RBCs and subsequent release of insulin. FIG. 7B shows percentages of insulin⁺ RBCs. C57BL/6 mice were i.v. injected with AAM or PBS twice daily for three days. After 14 days, RBCs were isolated and incubated with DBCO-insulin for 1 h. Cell-surface insulin was detected by staining with rabbit anti-insulin and Cy5-conjugated goat anti-rabbit secondary antibody. FIG. 7C is a schematic diagram for a type-1 diabetes study. AAM or PBS was i.v. injected into STZ-treated mice twice daily for three days, followed by i.p. injection of DBCO-insulin or insulin at four days post the last injection of AAM or PBS. FIG. 7D shows plasma insulin levels at 8 h post injection of DBCO-insulin or insulin. FIGS. 7E-7H show glucose tolerance test (GTT). Mice were fasted for 12 h, and then 10 IU/kg DBCO-insulin or insulin was i.p. injected. Glucose was i.p. injected at 3 and 6 h. FIG. 7E shows blood glucose levels of mice after the injection of DBCO-insulin or insulin. FIG. 7F shows accumulated blood glucose levels between 0-3 h, 3-6 h, and 6-9 h. FIG. 7G shows body weight of mice at 0, 2, and 4 weeks post the injection of DBCO-insulin or insulin. FIG. 7H shows percentile changes of mouse body weight over time for each group. The p-value for the statistical comparison between PBS+DBCO-insulin and AAM+DBCO-insulin groups is provided. All the numerical data are presented as mean±SD (two-tailed Welch's t-test was used for FIGS. 7B, 7E, 7F, 7G, and 7H; one-way ANOVA with post hoc Fisher's LSD test was used for FIG. 7D; 0.01<*P≤0.05; **P≤0.01; ***P≤0.001; ****P≤0.0001).

FIGS. 8A-8H illustrate metabolic glycan labeling of MEL cells. FIG. 8A shows mean Cy5 fluorescence intensity of MEL cells after incubation with azido-sugars or PBS for varied time (1, 3, 6, 12, 24, or 48 h) and staining with DBCO-Cy5 for 1 h. Concentration of 50 μM was used for all azido-sugars (AAM, AAGal, or AAGlu). FIG. 8B shows Cy5 fluorescence intensity ratio of MEL cells (azido-sugar/PBS) after incubation with azido-sugars for 1, 3, 6, 12, 24, and 48 h, respectively. FIG. 8C shows fluorescence images of MEL cells treated with AAM for 24 h and then incubated with DBCO-Cy5 for 1 h. Scale bar represents 10 μm. For FIGS. 8D-8G, MEL cells were treated with different concentrations of AAM for 24, 48 and 72 h, respectively, with fresh AAM added at 48 h. Shown are % Cy5⁺ MEL cells (FIG. 8D), mean Cy5 fluorescence of MEL cells (FIG. 8E), Cy5 fluorescence ratio of MEL cells (AAM/PBS) (FIG. 8F), and counts of MEL cells after azido-sugar treatment (FIG. 8G). FIG. 8H shows size of cells during the differentiation process of MEL cells in the presence or absence of AAM. All the numerical data are presented as mean±SD (one-way ANOVA with post hoc Fisher's LSD test was used; 0.01<*P≤0.05; **P≤0.01; ***P≤0.001; ****P≤0.0001).

FIGS. 9A-9E illustrate metabolic glycan labeling of mouse RBCs in vitro. FIG. 9A is a schematic illustration of metabolic labeling of mouse RBCs with azido groups and subsequent conjugation of DBCO-molecules via click chemistry. FIG. 9B shows % Cy5⁺ RBCs and FIG. 9C shows the mean Cy5 fluorescence intensity of RBCs after 24 h incubation with AAM or PBS, and 1 h staining with DBCO-Cy5. FIG. 9D shows representative phosphatidylserine (PS) histogram of mouse RBCs after 24 h incubation with AAM or AAGal or PBS. FIG. 9E shows % PS⁺ RBCs after 24 h incubation with AAM or AAGal or PBS. All the numerical data are presented as mean±SD (one-way ANOVA with post hoc Fisher's LSD test was used; 0.01<*P≤0.05; **P≤0.01; ***P≤0.001; ****P≤0.0001).

FIGS. 10A-10F illustrate that AAM can metabolically tag glycoproteins and glycolipids of RBCs. C57BL/6 mice were i.v. injected with AAM or PBS twice daily for three days. FIG. 10A shows Cy5 fluorescence intensity of RBCs and WBCs that were harvested at different times and stained with DBCO-Cy5. FIG. 10B shows the % RBCs and WBCs in the blood at different times post injections of AAM or PBS. FIG. 10C shows the representative flow plots for analyzing azido-labeled RBCs. FIG. 10D shows Western blot analysis of RBCs isolated from mice at 14 days post AAM or PBS injections. Proteins extracted from RBC membranes were run on a gel, incubated with DBCO-biotin, and detected with streptavidin-horseradish peroxidase (HRP) conjugate and chemiluminescence imaging. FIG. 10E shows quantification of protein band signals in FIG. 10D. FIG. 10F shows quantification of Cy5-conjugated lipids from HPLC profiles (shown in FIG. 2D). Lipids from RBCs were stained with DBCO-Cy5 and run on HPLC. All the numerical data are presented as mean±SD (two-tailed Welch's t-test was used; 0.01<*P≤0.05; **P≤0.01; ***P≤0.001; ****P≤0.0001).

FIGS. 11A-11C illustrate that intravenously injected AAM successfully labels RBC precursor cells in the bone marrow. C57BL/6 mice were i.v. injected with AAM or PBS twice daily for three days. Bone marrow was harvested at 48 h post injections of AAM or PBS. FIG. 11A shows representative flow cytometric plots used for analyzing different erythroid lineage cells in the bone marrow. FIG. 11B shows the percentages of different RBC precursor cells in the bone marrow at 2 days post injections of AAM or PBS. FIG. 11C represents the percentages of nucleated erythroid lineage cells in the bone marrow at 2 days post injections of AAM or PBS. All the numerical data are presented as mean±SD (one-way ANOVA with post hoc Fisher's LSD test was used; 0.01<*P≤0.05; **P≤0.01; ***P≤0.001; ****P≤0.0001).

FIGS. 12A-12D shows that intraperitoneally injected AAM metabolically labels RBCs with azido groups in vivo in a dose frequency dependent manner. FIG. 12A shows a schematic diagram illustrating the in vivo RBC labeling study. AAM (200 mg/kg) or PBS was intraperitoneally injected for 0, 2, 4, and 6 times at 12-h interval. RBCs were then isolated at different time points and incubated with DBCO-Cy5 for the detection of cell-surface azido groups. FIG. 12B shows the percentages of Cy5⁺ RBCs at 1 or 3 days post the last injection of AAM or PBS. FIG. 12C shows mean Cy5 fluorescence intensity of RBCs at 1 or 3 days post the last injection. FIG. 12D shows Cy5 fluorescence intensity ratio of RBCs (AAM/PBS) in FIG. 12C. All the numerical data are presented as mean±SD (one-way ANOVA with post hoc Fisher's LSD test was used; 0.01<*P≤0.05; **P≤0.01; ***P≤0.001; ****P≤0.0001).

FIGS. 13A-13C show that DBCO-Cy5 conjugated to azido-labeled RBCs shows stable membrane retention. For FIGS. 13A-13C, AAM or PBS was i.v. injected into C57BL/6 mice twice daily for three days. After 7 days, RBCs were isolated and incubated with DBCO-Cy5 for 1 h ex vivo. After washing, RBCs were stored in the Alsever's solution for different times, prior to flow cytometry analysis. FIG. 13A shows representative fluorescence images of RBCs isolated from AAM- or PBS-treated mice. Scale bar represents 100 μm. FIG. 13B shows Cy5 fluorescence intensity of RBCs over time. FIG. 13C shows Cy5 fluorescence intensity ratio of RBCs (AAM/PBS) in FIG. 13B. All the numerical data are presented as mean±SD (one-way ANOVA with post hoc Fisher's LSD test was used; 0.01<*P≤0.05; **P≤0.01; ***P≤0.001; ****P≤0.0001).

FIGS. 14A-14F illustrate in vivo conjugation of DBCO-Cy5 onto RBCs enables long-term fluorescence imaging of blood vessels and tissues. For FIG. 14A, AAM or PBS was i.v. injected into Balb/c mice twice daily for three days (day −9.5 to day −7), followed by subcutaneous injection of 4T1 tumor cells on day −3 and i.p. injection of DBCO-Cy5 on day 0. FIG. 14A shows the IVIS images of spleens harvested from mice at 21 days post injection of DBCO-Cy5. The terminal arteriole structures in the spleen are indicated by the arrows. FIG. 14B shows a schematic illustration of the RBC labeling and B16F10 tumor imaging study. AAM or PBS was i.v. injected twice daily for three days, followed by i.v. injection of DBCO-Cy5 on day 0 and subcutaneous injection of B16F10 tumor cells on day 17. FIG. 14C shows IVIS imaging of B16F10 tumors at 7 days post tumor inoculation. FIG. 14D represents quantified Cy5 fluorescence intensity of B16F10 tumors in FIG. 14C. DBCO-Cy5 was i.v. injected at 4 days post the last injection of AAM or PBS. Mouse organs were harvested at 24 days post injection of DBCO-Cy5 and imaged via the IVIS system. FIG. 14E shows IVIS imaging of major organs harvested from AAM- or PBS-treated mice on day 24. FIG. 14F shows quantified Cy5 fluorescence intensity of organs from FIG. 14E. All the numerical data are presented as mean±SD (two-tailed Welch's t-test was used; 0.01<*P≤0.05; **P≤0.01; ***P≤0.001; ****P≤0.0001).

FIGS. 15A-15D illustrate synthesis and characterization of DBCO-DOTA-Gd. FIG. 15A is a schematic showing synthesis route of DBCO-DOTA-Gd. FIG. 15B shows the ¹H NMR spectra of DBCO-NH₂, DOTA, and DBCO-DOTA in D₂O. FIG. 15C shows HPLC profiles of DBCO-NH₂, DBCO-DOTA, and DBCO-DOTA-Gd. The detection wavelength was set at 310 nm. FIG. 15D shows mass spectrum data of DBCO-DOTA-Gd. The peaks for [M+H]⁺, [M+2H]²⁺ and [M+Na]⁺ can be found.

FIGS. 16A-16H illustrate that azido-tagged RBCs enable conjugation of small-molecule and macromolecular cargos in vitro and in vivo. FIG. 16A shows acid-labile DBCO-doxorubicin (DBCO-Dox). FIG. 16B shows the release kinetics of DBCO-Dox at different pH, as measured by HPLC. For FIGS. 16C-16D, C57BL/6 mice were i.v. injected with AAM twice daily for three days, and DBCO-Dox was i.v. injected at 4 days post the last AAM injection. The blood was collected after 2 or 24 h post DBCO-Dox injection. FIG. 16C is a schematic of the study. FIG. 16D shows the percentages of Dox-containing RBCs at 2 or 24 h post DBCO-Dox injection. FIG. 16E shows the UV spectra of PE and DBCO-functionalized PE. FIGS. 16F-16H illustrate flow cytometry analysis of in vitro conjugation of PE to RBCs. RBCs were collected from AAM-treated mice at 14 days post AAM injection and incubated with DBCO-PE for 2 h. FIG. 16F shows a representative PE histogram of RBCs. FIG. 16G shows percentages of PE-containing RBCs. FIG. 16H shows the mean PE fluorescence intensity of RBCs. All the numerical data are presented as mean±SD (two-tailed Welch's t-test was used; 0.01<*P≤0.05; **P≤0.01; ***P≤0.001; ****P≤0.0001).

FIGS. 17A-17F illustrate that DBCO-insulin can conjugate to azido-labeled RBCs in vivo for improved blood glucose control. FIG. 17A illustrates synthesis of DBCO-insulin with an ester linkage. FIG. 17B shows HPLC profiles of insulin and DBCO-insulin with a detection wavelength of 280 nm. Peaks for DBCO-insulin are indicated. For FIGS. 17C and 17D, 4T1 cells were treated with AAM or PBS for 24 h, followed by 1 h incubation with DBCO-insulin. Cell-surface insulin was detected by staining with rabbit anti-insulin and Cy5-conjugated goat anti-rabbit secondary antibody. Shown are percentages of insulin-positive 4T1 cells (FIG. 17C) and mean Cy5 fluorescence intensity of 4T1 cells (FIG. 17D). FIG. 17E shows the mean Cy5 fluorescence intensity of RBCs. C57BL/6 mice were i.v. injected with AAM or PBS twice a day for three days. After 14 days, RBCs were isolated and incubated with DBCO-insulin for 1 h. Cell-surface insulin was detected by staining with rabbit anti-insulin and Cy5-conjugated goat anti-rabbit secondary antibody. FIG. 17F shows blood glucose levels of individual mice for each group during the course of the glucose tolerance test. Mice were fasted for 12 h, and then 10 IU/kg DBCO-insulin or insulin was i.p. injected. Glucose was i.p. injected at 3 and 6 h. All the numerical data are presented as mean±SD (two-tailed Welch's t-test was used; 0.01<*P≤0.05; **P≤0.01; ***P≤0.001; ****P≤0.0001).

### SEQUENCES

Any nucleic acid and amino acid sequences listed herein are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

SEQ ID NOs: 1 and 2 are exemplary autoantigen peptides.

### DETAILED DESCRIPTION

Disclosed herein is a RBC tagging and targeting technology that enables metabolic labeling of RBCs with chemical tags for subsequent targeted conjugation of diagnostic and therapeutic agents via efficient click chemistries. It is demonstrated that unnatural azido-sugars can metabolically label RBCs with azido groups via metabolic glycoengineering processes in vitro and in vivo, and it is further demonstrated that the azido-labeled RBCs can mediate targeted conjugation of dibenzocyclooctyne (DBCO)-bearing agents via the click reaction between azide and DBCO. This RBC tagging and targeting technology not only enables the development of improved therapies for treating diseases, but also provides a unique platform for studying RBC metabolism and immunology.

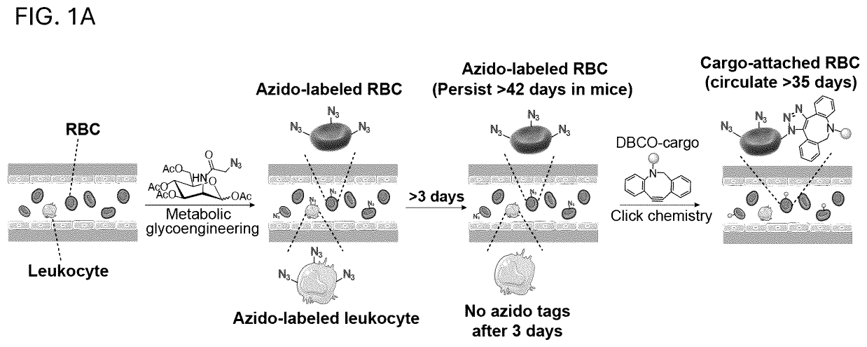

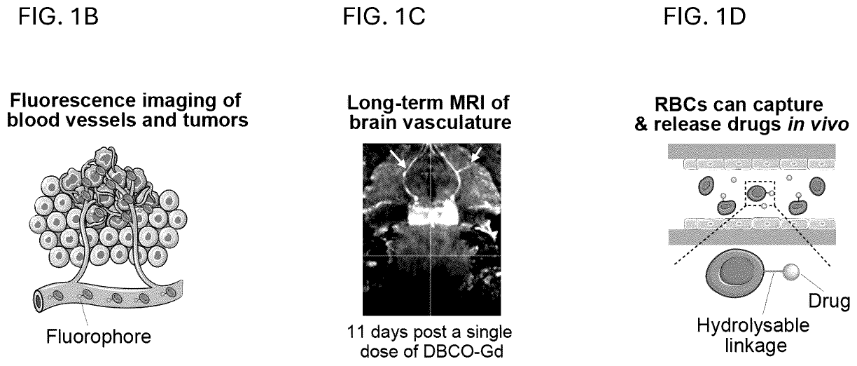

The ability to metabolically label RBCs with chemical tags holds tremendous promise for various applications including, for example drug delivery. Drugs can be efficiently conjugated onto circulating, chemically tagged RBCs for prolonged blood circulation and enhanced accumulation in target tissues such as tumors (e.g., FIG. 1B). The compositions and methods disclosed herein can be generally applied to different types of drugs and tumors. The disclosed compositions and methods can also be used in methods of immune tolerance induction. For example, autoantigens can be conjugated onto circulating, chemically tagged RBCs for subsequent phagocytosis by macrophages and induction of antigen-specific tolerance against autoimmune diseases. In addition, the disclosed compositions and methods can be used for imaging analysis, such as magnetic resonance imaging (MRI) or in vivo optical imaging (IVIS).

### I. Terms

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a cell” includes single or plural cells and is considered equivalent to the phrase “comprising at least one cell.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

In order to facilitate review of the disclosure, the following explanations of specific terms are provided.

Autoimmune disease or disorder: A disease or condition in which the immune system responds to self-antigens (autoreactive immune cells) resulting in self-destruction of healthy tissue. Examples of autoimmune disease or disorders include rheumatoid arthritis, systemic lupus erythematosus, type 1 diabetes, multiple sclerosis (or its animal model experimental autoimmune encephalomyelitis (EAE)), Sjögren's syndrome, Graves' disease, myasthenia gravis, ulcerative colitis, Hashimoto's thyroiditis, celiac disease, Crohn's disease, arthritis, inflammatory bowel disease, or scleroderma.

Click chemistry: A category of chemical reactions typically used to join a molecule of interest with a specific biomolecule. Click reactions occur in a single vessel, are not disturbed by water, generate minimal and non-toxic byproducts, and are characterized by a high thermodynamic driving force that drives it quickly and irreversibly to high yield of a single reaction product, with high reaction specificity. Click chemistry reactions include 3+2] cycloadditions (e.g., Huisgen 1,3-dipolar cycloaddition), thiol-ene reaction, Diels-Alder reaction, [4+1] cycloaddition between isonitrile and tetrazine, azide-alkyne reactions, tetrazine-norbornene reactions, tetrazine-cyclooctene reactions, or maleimide-thiol reactions.

Covalently coupled, conjugated, or linked: Coupling a first unit to a second unit. This includes, but is not limited to, covalently bonding one molecule to another molecule (for example, directly or via a linker molecule), noncovalently bonding one molecule to another (e.g. electrostatically bonding), non-covalently bonding one molecule to another molecule by hydrogen bonding, non-covalently bonding one molecule to another molecule by van der Waals forces, and any and all combinations of such couplings. In one embodiment, conjugating includes covalent bond linkage of a glycoprotein (such as a glycoprotein including a non-naturally occurring sugar moiety on a RBC) to a cargo. The covalent bond linkage can be direct or indirect, e.g., linked though a spacer molecule or other linker molecule. In some examples, the linkage includes a pH-labile or hydrolysable ester moiety, or other component that allows release of the cargo.

Inhibiting or treating a disease: Inhibiting the full development of a disease or condition, for example, in a subject who is at risk for a disease, such as a subject with cancer, an autoimmune disease, or an infection (such as a bacterial or viral infection). “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology.

Isolated: An “isolated” or “purified” biological component (such as a cell, nucleic acid, peptide, protein) has been substantially separated, produced apart from, or purified away from other components (for example, other biological components in the cell or environment in which the component occurs). Cells (such as RBCs), nucleic acids, peptides and proteins that have been “isolated” or “purified” thus include cells (such as RBCs), nucleic acids, or proteins purified by standard purification methods.

The term “isolated” or “purified” does not require absolute purity; rather, it is intended as a relative term. Thus, for example, an isolated biological component is one in which the biological component is more enriched than the biological component is in its natural environment within a cell, organism, sample, or production vessel (for example, a cell culture system). Preferably, a preparation is purified such that the biological component represents at least 50%, such as at least 70%, at least 80%, at least 90%, at least 95%, or greater, of the total biological component content of the preparation.

Pharmaceutically acceptable carrier: Remington: The Science and Practice of Pharmacy, The University of the Sciences in Philadelphia, Editor, Lippincott, Williams, & Wilkins, Philadelphia, PA, 21^{st} Edition (2005), describes compositions and formulations suitable for pharmaceutical delivery of one or more therapeutic compounds or molecules, such as one or more disclosed RBC preparations, and/or additional pharmaceutical agents.

Purified: The term purified does not require absolute purity; rather, it is intended as a relative term. Thus, for example, a purified RBC preparation is one in which the RBC is more enriched than in its original environment. In one embodiment, a preparation is purified such that a component (such as purified RBCs) represents at least 50% of the total content of the preparation. A substantially purified RBC preparation is at least 60%, 70%, 80%, 90%, 95% or 98% pure. Thus, in one specific, non-limiting example, a substantially purified RBC preparation is 90% free of other components.

Red blood cell (RBC): Also referred to as erythrocytes, RBCs are the most common type of blood cell, making up 40-45% of whole blood volume and >99% of blood cells. They are responsible for delivering oxygen to the tissues, via binding and release of oxygen to hemoglobin present in the cells. In mammals, mature RBCs lack a nucleus and organelles and their cytoplasm is primarily hemoglobin. The cell membrane of RBCs includes cholesterol and phospholipids, as well as glycoproteins, including the blood group antigens. RBCs have a long half-life (˜120 days in humans and ˜45 days in mice) and are recycled by macrophages.

As used herein, a modified RBC refers to a RBC labeled with an azido-sugar (such as having surface proteins or lipids including an unnatural azido-containing moiety), a RBC having surface proteins labeled with an azido-sugar and conjugated or covalently linked to a cargo, or both. In some examples, a modified RBC includes one or more surface proteins or lipids covalently linked to a cargo by click chemistry. The modified RBC may be in vitro, ex vivo, or in vivo.

Subject: As used herein, the term “subject” refers to a mammal and includes, without limitation, humans, domestic animals (e.g., dogs or cats), farm animals (e.g., cows, horses, or pigs), and laboratory animals (mice, rats, hamsters, guinea pigs, pigs, rabbits, dogs, or monkeys). In some embodiments the subject has a disease or disorder, such as cancer, an autoimmune disease, or an infection.

Therapeutically effective amount: The quantity of an agent (e.g., modified RBCs), that is sufficient to treat, reduce, and/or ameliorate the symptoms and/or underlying cause of a disease or pathological condition, such as cancer, an autoimmune disorder, or an infection in a subject. In a specific non-limiting example, an effective amount is an amount sufficient to inhibit or reduce tumor growth in the subject. In another specific non-limiting example, an effective amount is an amount sufficient to inhibit or reduce inflammation in the subject. In a further example, an effective amount is an amount sufficient to reduce or inhibit an infection in the subject.

Treating, treatment, and therapy: Any success or indicia of success in the attenuation or amelioration of a disease or disorder, including any objective or subjective parameter such as abatement, remission, diminishing of symptoms or making the condition more tolerable to the patient, slowing in the rate of progression, degeneration or decline, making the final point of degeneration less debilitating. The treatment may be assessed by objective or subjective parameters; including the results of a physical examination, neurological examination, or psychiatric evaluations. For example, treatment of a cancer can include decreasing the size, volume, or weight of a tumor, decrease the number, size, volume, or weight of metastases, or combinations thereof.

Tumor, neoplasia, malignancy or cancer: A neoplasm is an abnormal growth of tissue or cells which results from excessive cell division. Neoplastic growth can produce a tumor. The amount of a tumor in an individual is the “tumor burden,” which can be measured as the number, volume, or weight of the tumor. A tumor that does not metastasize is referred to as “benign.” A tumor that invades the surrounding tissue and/or can metastasize is referred to as “malignant.” A “non-cancerous tissue” is a tissue from the same organ wherein the malignant neoplasm formed, but does not have the characteristic pathology of the neoplasm. Generally, noncancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer.

### II. Modified Red Blood Cells

Provided herein are modified red blood cells that include one or more surface proteins or lipids covalently linked to a cargo, which in particular examples is via click chemistry. These modified RBCs can be used in methods of treating a disease or disorder (including cancer, an autoimmune disease, a bacterial or viral infection, or other disorder).

In some examples, RBCs that include one or more surface proteins or lipids that are covalently linked to a cargo via click chemistry are provided. In some examples, the cargo is covalently linked to the one or more proteins or lipids indirectly, for example, through a glycan moiety (for example, the cargo is linked to a glycoprotein or glycolipid). In particular examples, the glycan moiety includes a linker (or chemical tag) that can be used in conjugating the cargo to the protein or lipid. In some examples, the RBCs are from a subject with a disease or disorder (such as cancer, an autoimmune disease, or an infection). In particular examples, the RBCs are from a human subject.

In some examples, the cargo is covalently linked to the glycoprotein or glycolipid via an azide group incorporated in a non-naturally occurring sugar in the protein. In some examples, the azide group is incorporated into the glycoprotein via metabolic labeling of cells with acetylated N-azidoacetyl-D-mannosamine (Ac₄ManNAz, also referred to as AAM), tetra-acetylated N-azidoacetyl-D-galactosamine (Ac₄GalNAz), tetra-acetylated N-azidoacetyl-D-glucosamine (Ac₄GlcNAz), N-azidoacetyl-D-mannosamine (ManNAz), N-azidoacetyl-D-galactosamine (GalNAz), N-azidoacetyl-D-glucosamine (GlcNAz), or 9-azido-9-deoxy-N-acetylneuraminic acid (9AzNeu5Ac). In other examples, the cargo is covalently linked to a glycoprotein or glycolipid via a chemical tag or linker selected from diazoalkane, cyclopropene, isonitrile, alkene, diazirine, DBCO, alkyne, or ketone (e.g., as described in Wang and Mooney, Nature Chemistry, 12:1102-1114, 2020, incorporated herein by reference in its entirety). The chemical tag or linker can be incorporated into a glycoprotein or glycolipid via metabolic labeling of cells with N-modified mannosamine, 6-modified fucose, N-modified galactosamine, N-modified glucosamine, or 9-modified 9-deoxy-N-acetylneuraminic acid (or named 9-modified sialic acid). The cargo can be linked to the labeled RBC surface protein or lipid using click chemistry, such as azide-alkyne click chemistry, tetrazine-norbornene click chemistry, tetrazine-cyclooctene click chemistry, or maleimide-thiol click chemistry.

The disclosed methods can be used to conjugate or link RBCs with a wide variety of cargos, such as therapeutic agents or immunogenic agents. In some examples, the cargo is capable of covalent binding to the one or more azido-labeled surface proteins or lipids by click chemistry, such as azide-alkyne click chemistry. For example, the cargo may include or is linked to a dibenzocyclooctyne (DBCO) moiety, for linking to the azido-labeled surface proteins or lipids. In some examples, the cargo may further include a linker that allows release of the active portion of the cargo in the blood. The linker is between the DBCO moiety and the cargo. In some examples, the cargo includes a pH-labile linkage. In other examples, the cargo includes a hydrolysable ester linkage. In other examples, the cargo includes a disulfide linkage. In further examples, the cargo includes a peptide linker that can be cleaved by an enzyme, for example a peptide that can be cleaved by a cathepsin (such as Val-Cit), a matrix metalloproteinase, thrombin, a caspase, or fibroblast activation protein. Exemplary cleavage sites are described in Poreba, FEBS J. 287:1936-1969, 2020.

In some examples, the cargo is an autoantigen, for example, for use in treating or inhibiting an autoimmune disorder. In particular examples, the autoantigen is a protein or peptide that is associated with an autoimmune disorder. Exemplary autoantigens include a BDC peptide (e.g., AAVRPLWVRMEAA; SEQ ID NO: 1), which is associated with type 1 diabetes, or a MOG35-55 peptide (e.g., MEVGWYRSPFSRVVHLYRNGK; SEQ ID NO: 2), which is associated with experimental autoimmune encephalomyelitis (EAE), an animal model of multiple sclerosis. One of ordinary skill in the art can select an appropriate autoantigen for a particular disorder.

In other examples, the cargo is a compound for the treatment of cancer. Exemplary cancer therapeutic agents include, but are not limited to alkylating agents, such as nitrogen mustards (for example, chlorambucil, chlormethine, cyclophosphamide, ifosfamide, and melphalan), nitrosoureas (for example, carmustine, fotemustine, lomustine, and streptozocin), platinum compounds (for example, carboplatin, cisplatin, oxaliplatin, and BBR3464), busulfan, dacarbazine, mechlorethamine, procarbazine, temozolomide, thiotepa, and uramustine; antimetabolites, such as folic acid (for example, methotrexate, pemetrexed, and raltitrexed), purine (for example, cladribine, clofarabine, fludarabine, mercaptopurine, and thioguanine), pyrimidine (for example, capecitabine), cytarabine, fluorouracil, and gemcitabine; plant alkaloids, such as podophyllum (for example, etoposide, and teniposide), taxane (for example, docetaxel and paclitaxel), vinca (for example, vinblastine, vincristine, vindesine, and vinorelbine); cytotoxic/antitumor antibiotics, such as anthracycline family members (for example, daunorubicin, doxorubicin, epirubicin, idarubicin, mitoxantrone, and valrubicin), bleomycin, hydroxyurea, and mitomycin; topoisomerase inhibitors, such as topotecan, camptothecin, and irinotecan; monoclonal antibodies, such as alemtuzumab, bevacizumab, cetuximab, daratumumab, elotuzumab, gemtuzumab, rituximab, panitumumab, atezolizumab, avelumab, ipilimumab, ofatumumab, nivolumab, pembrolizumab, rituximab, durvalumab, and trastuzumab; photosensitizers, such as aminolevulinic acid, methyl aminolevulinate, porfimer sodium, and verteporfin; proteasome inhibitors, such as bortezomib, carfilzomib, oprozomib, ixazomib, marizomib, and delanzomib; kinase inhibitors, such as gefitinib, imatinib, sunitinib, sorafenib, vemurafenib, trametinib, and ruxolitinib; growth factor receptor inhibitors, such as acitinib, erlotinib, cabozantinib, and crizotinib; mTOR inhibitors, such as everolimus, temsirolimus, and temisorotimus; and other agents, such as alitretinoin, altretamine, amsacrine, anagrelide, arsenic trioxide, asparaginase, bexarotene, celecoxib, denileukin diftitox, enzalutamide, flutamide, nilutamide, bicalutamide, topilutamide, apalutamide, estramustine, hydroxycarbamide, pentostatin, masoprocol, mitotane, pegaspargase, tamoxifen, clomifene, raloxifene, anastrozole, fulvestrant, and tretinoin. In particular non-limiting examples, the cargo is doxorubicin, paclitaxel, camptothecin, anti-PD-1 (such as pembrolizumab, nivolumab, or cemiplimab), anti-PDL1 (such as atezolizumab, avelumab, or durvalumab), or anti-CTLA-4 (such as ipilimumab or tremelimumab). One of ordinary skill in the art can select an appropriate cancer therapeutic agent for a particular subject and cancer, from those listed above, or additional agents.

In further examples, the cargo is an antigen, for example an antigen that can induce an immunogenic response in a subject. In some examples, the antigen is a bacterial or viral antigen. An exemplary antigen is a coronavirus antigen, such as a spike protein from coronavirus (for example, SARS-CoV-2 spike protein). One of ordinary skill in the art can select an appropriate antigen, for example, for vaccination against a particular bacteria or virus.

In additional examples, the cargo is an anti-inflammatory drug, such as celecoxib, diflunisal, etodolac, fenoprofen, flurbiprofen, indomethacin, ketoprofen, ketorolac, mefenamic acid, meloxicam, nabumetone, oxaprozin, piroxicam, sulindac, tolmetin, diclofenac, ibuprofen, or naproxen.

In further examples, the cargo is a peptide. In one example, the cargo is insulin.

In additional examples, the cargo is an antibiotic or an antiviral agent. Exemplary antibiotics include but are not limited to amoxicillin, azithromycin, cephalexin, doxycycline, sulfamethoxazole, trimethoprim, metronidazole, mupirocin, cefdinir, vancomycin, clindamycin, ciprofloxacin, levofloxacin, moxifloxacin, linezolid, tedizolid, and tigecycline. One of ordinary skill in the art can select an appropriate antibiotic, for example, depending on the bacterial infection to be treated. Exemplary antiviral agents include but are not limited to nirmatrelvir, ritonavir, oseltamivir, entecavir, tenofovir, sofosbuvir, velpatasvir, ledipasvir, glecaprevir, pibrentasivr, voxilaprevir, grazoprevir, valavyclovir, famciclovir, acyclovir, ganciclovir, foscarnet, emtricitabine, lamivudine, zidovudine, abacavir, rilpivirine, etravirine, doravirine, nevirapine, tipranavir, fosamprenavir, ritonavir, darunavir, atazanavir, enfuviritide, maraviroc, raltegravir, dolutegravir, cabotegravir, fostemsavir, and lenacapavir. One of ordinary skill in the art can select an appropriate antiviral, for example, depending on the viral infection to be treated.

In further examples, the cargo is an antibody or a fragment thereof, such as a monoclonal antibody, a bispecific antibody, or an scFv. The antibody or fragment thereof can be a therapeutic antibody (such as a cancer therapeutic antibody) or a neutralizing antibody (such as neutralizing antibody against a bacterial or viral pathogen).

Also provided are compositions including the modified RBCs and a pharmaceutically acceptable carrier. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. In addition to biologically neutral carriers, pharmaceutical compositions to be administered can contain minor amounts of non-toxic auxiliary substances, such as wetting or emulsifying agents, preservatives, and pH buffering agents and the like, for example, sodium acetate or sorbitan monolaurate. In particular examples, the carrier may be sterile, and/or suspended or otherwise contained in a unit dosage form containing one or more measured doses of the composition suitable to treat or inhibit a disease or disorder (such as cancer). It may also be accompanied by medications for its use for treatment purposes. The unit dosage form may be, for example, in a sealed vial that contains sterile contents or a syringe for injection into a subject, or lyophilized for subsequent solubilization and administration or in a solid or controlled release dosage.

### III. Methods of Treating a Subject

Methods of treating a subject with a disease or disorder with the modified RBCs described herein are provided. In some examples, the modified RBCs are administered to the subject to treat the disease or disorder. In other examples, the subject is administered an azido-modified sugar and a cargo capable of covalently binding to one or more of the resulting azido-labeled surface proteins or lipids on the surface of RBCs in the subject. In particular examples, the subject is a human subject.

In some examples, the methods are in vivo methods and include administering to a subject a composition including an azido-modified sugar moiety, thereby generating red blood cells comprising one or more azido-labeled surface proteins; and administering to the subject a composition including a cargo for treating or inhibiting the disease, wherein the cargo is capable of covalently binding to the one or more azido-labeled surface proteins or lipids. In some examples, the composition including an azido-modified sugar moiety is administered to the subject one or more times (such as 1, 2, 3, 4, 5, 6, or more times). If the composition including an azido-modified sugar moiety is administered more than one time, it is administered about every 6 to 48 hours, such as about every 6 hours, about every 12 hours, about every 18 hours, about every 24 hours, about every 36 hours or about every 48 hours. In some examples, the composition including an azido-modified sugar moiety is administered to the subject about every 24 hours.

The cargo for treating or inhibiting the disease in some examples, is administered to the subject after the composition comprising an azido-modified sugar moiety. For example, the cargo may be administered to the subject after a sufficient time for RBCs in the subject to express one or more azido-labeled surface proteins, for example, about 3 hours to about 120 days after administering the composition including the azido-modified sugar moiety. In some examples, the cargo is administered to the subject about 3 hours, about 6 hours, about 12 hours, about 18 hours, about 1 day, about 2 days, about 3 days, about 4 days, about 5 days, about 6 days, about 7 days, about 10 days, about 2 weeks, about 3 weeks, about 4 weeks, about 5 weeks, about 6 weeks, about 7 weeks, about 8 weeks, about 9 weeks or about 10 weeks after administration of the azido-modified sugar moiety. In some examples, the cargo is administered to the subject at least about 3 days after administration of the azido-modified sugar moiety, such as at least about 3 days or about 4 days after administration of the azido-modified sugar moiety. In other examples, the cargo is administered to the subject prior to administering the one or more azido-modified sugar moiety, such as about 3 hours, about 6 hours, about 12 hours, about 18 hours, about 1 day, about 2 days, about 3 days, about 4 days, about 5 days, about 6 days, or about 7 days before administering the azido-modified sugar moiety.

In some examples, the composition including the azido-modified sugar moiety is administered to the subject intravenously. In some examples, the composition including the cargo is administered to the subject intravenously. In other examples, the composition including the cargo can be administered intraperitoneally, subcutaneously or intramuscularly.

In some examples, the subject is administered about 5-200 mg/kg of the azido-modified sugar (such as about 5-20 mg/kg, about 10-50 mg/kg, about 40-75 mg/kg, about 60-100 mg/kg, about 80-120 mg/kg, about 110-150 mg/kg, about 140-175 mg/kg, or about 160-200 mg/kg). In some examples, the amount of cargo administered to the subject includes the amount that is typically administered clinically to a subject with the disorder in other contexts. A skilled clinician can identify an appropriate amount of both the azido-modified sugar and the cargo to be administered to a subject, based on the disorder being treated, the condition of the subject, and other factors.

In other examples, the methods are ex vivo methods and include contacting a preparation of red blood cells obtained from a subject with a disease or disorder with a composition including an azido-modified sugar for a sufficient period of time for the red blood cells to express one or more azido-labeled surface proteins or lipids and contacting the red blood cells expressing the one or more azido-labeled surface proteins or lipids with a composition including a cargo for treating or inhibiting the disease, wherein the cargo is capable of covalently binding to the one or more azido-labeled surface proteins or lipids, thereby producing a population of modified red blood cells; and administering the population of modified red blood cells to the subject. In some examples, the RBCs are contacted with the azido-modified sugar moiety for about 1 h to about 120 h (such as about 1 h, about 2 h, about 3 h, about 4 h, about 5 h, about 6 h, about 8 h, about 12 h, about 18 h, about 24 h, about 36 h, about 48 h, about 60 h, about 72 h, about 84 h, about 96 h, about 108 h, or about 120 h) before contacting the with the composition including the cargo.

In particular examples, the azido-labeled sugar moiety is tetra-acetylated N-azidoacetyl-D-mannosamine (Ac₄ManNAz), N-azidoacetyl-D-mannosamine (ManNAz), tetra-acetylated N-azidoacetyl-D-galactosamine (Ac₄GalNAz), N-azidoacetyl-D-galactosamine (GalNAz), tetra-acetylated N-azidoacetyl-D-glucosamine (Ac₄GlcNAz), N-azidoacetyl-D-glucosamine (GlcNAz), or N-Acetyl-9-azido-9-deoxy-neuraminic acid (9-AzNue5Ac), for example, Ac₄ManNAz.

In some examples, the cargo is capable of covalent binding to the one or more azido-labeled surface proteins or lipids by click chemistry, such as azide-alkyne click chemistry. For example, the cargo may include (for example, is conjugated or linked to) a dibenzocyclooctyne (DBCO) moiety, for linking to the azido-labeled surface proteins or lipids.

In some embodiments, subject being treated has cancer, such as a solid tumor or a hematological malignancy and the cargo includes a cancer therapeutic agent, as discussed in Section II. In additional examples, the subject being treated has metastatic cancer. Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytoma, sebaceous gland carcinoma, papillary carcinoma, papillary adenocarcinoma, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyrgioma, ependymoma, pinealoma, hemangioblastoma, acoustic neuroma, oligodendroglioma, meningioma, melanoma, neuroblastoma, and retinoblastoma). Examples of hematological malignancies include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), T-cell large granular lymphocyte leukemia, polycythemia vera, lymphoma, diffuse large B-cell lymphoma, Hodgkin's lymphoma, non-Hodgkin's lymphoma (indolent and high grade forms), mantle cell lymphoma, follicular cell lymphoma, multiple myeloma, Waldenstrom's macroglobulinemia, heavy chain disease, myelodysplastic syndrome, hairy cell leukemia and myelodysplasia. In particular embodiments, the subject has lymphoma, breast cancer (such as triple negative breast cancer), colon cancer, glioblastoma, pancreatic cancer, or melanoma.

In other examples, the subject being treated has an autoimmune disease or disorder and the cargo includes an autoantigen. In some examples, the subject has multiple sclerosis, rheumatoid arthritis, systemic lupus erythematosus, type 1 diabetes, multiple sclerosis, Sjögren's syndrome, Graves' disease, myasthenia gravis, ulcerative colitis, Hashimoto's thyroiditis, celiac disease, Crohn's disease, arthritis, inflammatory bowel disease, psoriasis, or scleroderma. In particular examples, the subject has type I diabetes, multiple sclerosis, or an animal model of multiple sclerosis, such as EAE.

In further examples, the subject being treated has diabetes (for example, type 2 diabetes) and the cargo is insulin.

In some examples of ex vivo methods, modified RBCs (such as RBCs including one or more proteins or lipids covalently linked to chemotherapeutic agent) are administered to a subject with cancer. In other examples, modified RBCs (such as RBCs including one or more proteins or lipids covalently linked to an autoantigen) are administered to a subject in need of, such as a subject who has an autoimmune disease or disorder. In further examples, modified RBCs (such as RBCs including one or more proteins or lipids covalently linked to a viral or bacterial protein) are administered to a subject in need thereof, such as a subject who has or is at risk of an infectious disease. The modified RBCs can be prepared from RBCs from the subject, for example, as described in Section IV. In some examples, an effective amount of modified RBCs can be administered to the subject. In some examples, the effective amount may be about 10⁵ to about 10⁹ modified RBCs (e.g., about 10⁵, about 10⁶, about 10⁷, about 10⁸, or about 10⁹ modified RBCs). The modified RBCs are administered intravenously. Multiple doses of the modified RBCs can be administered to the subject. For example modified RBCs can be administered daily, every other day, twice weekly, weekly, every other week, monthly, or less frequently. A skilled clinician can select an appropriate administration schedule based on the subject, the condition being treated, and other factors.

In examples of in vivo methods, the subject has cancer and is administered a composition including an azido-modified sugar moiety, thereby generating red blood cells with one or more azido-labeled surface proteins, and the is administered a composition including a cargo including a cancer therapeutic agent, wherein the cargo is capable of covalently binding to the one or more azido-labeled surface proteins or lipids. In further examples, the subject has an autoimmune disease or disorder, and is administered a composition including an azido-modified sugar moiety, thereby generating red blood cells with one or more azido-labeled surface proteins, and is administered a composition including a cargo including an autoantigen, wherein the cargo is capable of covalently binding to the one or more azido-labeled surface proteins or lipids. In still further examples, the subject has or is at risk of an infectious disease, and is administered a composition including an azido-modified sugar moiety, thereby generating red blood cells with one or more azido-labeled surface proteins, and is administered a composition including a cargo including an antigen of the infectious disease, wherein the cargo is capable of covalently binding to the one or more azido-labeled surface proteins or lipids. In additional examples, the subject has or is at risk of a bacterial infection, and is administered a composition including an azido-modified sugar moiety, thereby generating red blood cells with one or more azido-labeled surface proteins, and is administered a composition including a cargo including an antibiotic, wherein the cargo is capable of covalently binding to the one or more azido-labeled surface proteins or lipids. In other examples, the subject has or is at risk of a viral infection, and is administered a composition including an azido-modified sugar moiety, thereby generating red blood cells with one or more azido-labeled surface proteins, and is administered a composition including a cargo including an antiviral agent, wherein the cargo is capable of covalently binding to the one or more azido-labeled surface proteins or lipids.

### IV. Methods of Preparing Modified RBCs

Also provided are methods of preparing the disclosed modified RBCs. The methods utilize metabolic labeling of proteins or lipids in the RBC (such as surface proteins or lipids) with non-naturally occurring sugars.

In some examples, RBCs are labeled in vitro or ex vivo, while in other examples, RBCs are labeled in vivo. The metabolic labeling process utilizes non-naturally occurring sugars that can be incorporated into glycoproteins or glycolipids and can be used to covalently couple the glycoprotein or glycolipid to a molecule of interest via a chemical tag on the sugar, for example using click-chemistry methods. In some embodiments, the methods utilize an azido-acetylated sugar moiety that can be incorporated into a glycoprotein or glycolipid, such as tetra-acetylated N-azidoacetyl-D-mannosamine (Ac₄ManNAz), N-azidoacetyl-D-mannosamine (ManNAz), tetra-acetylated N-azidoacetyl-D-galactosamine (Ac₄GalNAz), N-azidoacetyl-D-galactosamine (GalNAz), tetra-acetylated N-azidoacetyl-D-glucosamine (Ac₄GlcNAz), N-azidoacetyl-D-glucosamine (GlcNAz), or N-Acetyl-9-azido-9-deoxy-neuraminic acid (9-AzNue5Ac). In one example, Ac₄ManNAz is taken up by RBCs, and is hydrolyzed by esterases, followed by phosphorylation and ring-opening isomerization and conversion to sialic acid by attack by phosphoenolpyruvic acid. The sialic acid is conjugated to a protein and expressed on the surface of an RBC in the form of a glycoprotein. In other embodiments, the azido-acetylated sugar moiety may as include a trigger-responsive moiety that is cleaved by a trigger (such as a trigger that is enhanced or increased in cancer cells) and a linker, such as a self-immolative linker. Exemplary trigger-responsive moieties are described in International Patent Application Publication No. WO 2017/062800, which is incorporated herein by reference in its entirety.

Thus, in some examples, the methods include culturing RBCs in vitro in the presence of an azido-labeled sugar moiety (such as 0.1-200 μM, for example, about 0.1-5 μM, about 1-10 μM, about 5-15 μM, about 10-25 μM, about 20-40 μM, about 30-50 μM, about 50-75 μM, about 60-100 μM, about 80-125 μM, about 100-150 μM, or about 150-200 μM). In one example, the cells are cultured with 50 μM azido-labeled sugar (such as 50 μM Ac4ManNAz) or 100 μM azido-labeled sugar (such as 100 μM Ac4ManNAz). In other embodiments, the cells are cultured in the presence of a non-naturally occurring sugar including N-modified mannosamine, 6-modified fucose, N-modified galactosamine, or N-modified glucosamine which are modified with a chemical tag selected from azide, diazoalkane, cyclopropene, isonitrile, alkene, diazirine, DBCO, alkyne, or ketone (e.g., as described in Wang and Mooney, Nature Chemistry, 12:1102-1114, 2020, incorporated herein by reference in its entirety). In some examples, the non-naturally occurring sugar is added to the culture medium one time, while in other examples, fresh medium including the non-naturally occurring sugar is added one or more times during the culture. In some examples, the RBCs are obtained or isolated from a subject, such as a subject with a disease or disorder (for example, a subject with cancer or an autoimmune disorder).

After a sufficient period of time in culture for incorporation of the non-naturally occurring sugar into cell surface proteins or lipids (for example, about 1 hour to about 120 hours, such as about 1 hour, about 2 hours, about 3 hours, about 4 hours, about 6 hours, about 8 hours, about 10 hours, about 12 hours, about 16 hours, about 18 hours, about 24 hours, about 36 hours, about 48 hours, about 60 hours, about 72 hours, about 84 hours, about 96 hours, about 108 hours, or about 120 hours), the labeled RBCs are then covalently coupled to a cargo (such as those described in Sections II and III). In particular examples, the cargo is covalently coupled to an azido-labeled RBC surface protein or lipid utilizing click chemistry, such as azide-alkyne click chemistry. Depending on the chemical tag included in the non-naturally occurring sugar, one of ordinary skill in the art can select appropriate click chemistry methods, including azido-alkyne click chemistry, tetrazine-norbornene click chemistry, tetrazine-cyclooctene click chemistry, or maleimide-thiol click chemistry.

In other examples, modified RBCs are prepared in vivo. In such examples, the methods of treating a subject as described in Section III may result in the preparation of modified RBC in vivo.

### V. Imaging Methods

In some embodiments, modified RBCs described herein can be utilized in imaging methods. In some examples, the methods can be used for imaging blood vessels in a subject or for imaging of tissues or tumors in a subject. In some examples, these methods are advantageous because a single dose of imaging agent is administered to a subject and imaging analysis can be carried out longitudinally, due to the persistence of the labeled RBCs.

In some examples, the methods include administering to a subject a composition including an azido-modified sugar moiety, thereby generating red blood cells comprising one or more azido-labeled surface proteins; administering to the subject a composition including an imaging agent, wherein the imaging agent is capable of covalently binding to the one or more azido-labeled surface proteins or lipids; and performing an imaging analysis of the subject. In some examples, the imaging analysis is carried out one or more times (such as 1, 2, 3, 4, 5, or more times). The imaging analysis may be carried out daily, every other day, twice weekly, weekly, every other week, or monthly, while the modified RBCs persist in the subject (for example, about 42 days in mice or about 120 days in humans).

The imaging agent capable of covalently binding to the one or more azido-labeled surface proteins or lipids in some examples is administered to the subject after the composition comprising an azido-modified sugar moiety. For example, the imaging agent capable of covalently binding to the one or more azido-labeled surface proteins or lipids may be administered to the subject after a sufficient time for RBCs in the subject to express one or more azido-labeled surface proteins, for example, about 3 hours to about 120 days after administering the composition including the azido-modified sugar moiety. In some examples, the imaging agent is administered to the subject about 3 hours, about 6 hours, about 12 hours, about 18 hours, about 1 day, about 2 days, about 3 days, about 4 days, about 5 days, about 6 days, about 7 days, about 10 days, about 2 weeks, about 3 weeks, about 4 weeks, about 5 weeks, about 6 weeks, about 7 weeks, about 8 weeks, about 9 weeks or about 10 weeks after administration of the azido-modified sugar moiety. In some examples, the imaging agent capable of covalently binding to the one or more azido-labeled surface proteins or lipids is administered to the subject at least about 3 days after administration of the azido-modified sugar moiety, such as at least about 3 days or about 4 days after administration of the azido-modified sugar moiety. In other examples, the imaging agent capable of covalently binding to the one or more azido-labeled surface proteins or lipids is administered to the subject prior to administering the one or more azido-modified sugar moiety, such as about 3 hours, about 6 hours, about 12 hours, about 18 hours, about 1 day, about 2 days, about 3 days, about 4 days, about 5 days, about 6 days, or about 7 days before administering the azido-modified sugar moiety.

In some examples, the composition including the azido-modified sugar moiety is administered to the subject intravenously. In some examples, the composition including the imaging agent is administered to the subject intravenously. In other examples, the composition including the imaging agent can be administered intraperitoneally, subcutaneously or intramuscularly.

In particular examples, the azido-labeled sugar moiety is tetra-acetylated N-azidoacetyl-D-mannosamine (Ac₄ManNAz), N-azidoacetyl-D-mannosamine (ManNAz), tetra-acetylated N-azidoacetyl-D-galactosamine (Ac₄GalNAz), N-azidoacetyl-D-galactosamine (GalNAz), tetra-acetylated N-azidoacetyl-D-glucosamine (Ac₄GlcNAz), N-azidoacetyl-D-glucosamine (GlcNAz), or N-Acetyl-9-azido-9-deoxy-neuraminic acid (9-AzNue5Ac), for example, Ac₄ManNAz.

In some examples, the imaging agent is capable of covalent binding to the one or more azido-labeled surface proteins or lipids by click chemistry, such as azide-alkyne click chemistry. For example, the imaging agent may include (for example, is conjugated or linked to) a dibenzocyclooctyne (DBCO) moiety, for linking to the azido-labeled surface proteins or lipids.

In some examples, the subject is a mammalian subject, such as a human, a domestic animal (e.g., dogs or cats), a farm animal (e.g., cows, horses, or pigs), or a laboratory animal (mice, rats, hamsters, guinea pigs, pigs, rabbits, dogs, or monkeys).

In some examples, the imaging agent is a fluorescent dye, such as Cy™5, Cy™3, or Cy™7 dye or a bioluminescent substrate, such as luciferin. Fluorescent dyes or bioluminescent substrates may be of use in methods utilizing domestic animals, farm animals, or laboratory animals. In such examples, the imaging method may be in vivo optical imaging (IVIS).

In other examples, the imaging agent is a contrast agent. In some examples, the contrast agent is a gadolinium-based contrast agent (GBCA). Exemplary GBCAs include gadopentetate dimeglumine (gadolinium diethylene triamine pentaacetic acid (Gd-DTPA), gadodiamide (gadolinium diethylene triamine penta-acetic acid bis-methylamide (GD-DTPA-BMA), Gadoteridol (Gadolinium-1,4,7-tris (carboxymethyl)-10-(2′ hydroxypropyl)-1, 4, 7-10-tetraazacyclododecane (Gd-HPD03A]), gadoterate meglumine (gadolinium-tetraazacyclododecane tetra acetic acid (Gd-DOTA), gadobenate dimeglumine; and gadobutrol. In other examples, the imaging agent is a superparamagnetic iron oxide (SPIO). Contrast agents may be used in magnetic resonance imaging methods, for example, with human subjects.

In other examples, the imaging agent is suitable for computed tomography methods, such as iohexol. In further examples, the imaging agent is suitable for positron emission tomography methods, such as DOTA-⁶⁴Cu or ¹⁸F-flouorodeoxyglucose. In still further examples, the imagining agent is suitable for ultrasound methods, such as microbubbles.

### EXAMPLES

The following examples are provided to illustrate certain particular features and/or implementations of the disclosure. These examples should not be construed to limit the disclosure to the particular features or implementations described.

### Example 1

### Materials and Methods

Materials and Instrumentation. Mannosamine hydrochloride, galactosamine hydrochloride, glucosamine hydrochloride, dicyclohexyl carbodiimide, N-hydroxysuccinimide, and other chemical reagents were purchased from Sigma Aldrich (St. Louis, MO, USA) unless otherwise noted. Primary antibodies used in this study were obtained from Thermo Fisher Scientific (Waltham, MA, USA). All antibodies were diluted according to the manufacturer's recommendations. Fixable viability dye efluor780 was obtained from Thermo Fisher Scientific (Waltham, MA, USA). SYTO™ 85 Orange Fluorescent Nucleic Acid Stain was purchased from Thermo Fisher Scientific (Waltham, MA, USA). ENLITEN® ATP Assay System Bioluminescence Detection Kit was purchased from Thermo Fisher Scientific (Waltham, MA, USA). Histopaque®-1077 was purchased from Sigma Aldrich (St. Louis, MO, USA). Halt™ Protease Inhibitor Cocktail was purchased from Thermo Fisher Scientific (Waltham, MA, USA). Nitrocellulose membrane was purchased from Thermo Fisher Scientific (Waltham, MA, USA). Insulin ELISA kit (Catalog 80-INSHU-E01.1) was purchased from ALPCO (Salem, NH, USA). Clarity BG1000 Blood Glucose Meter and Strips were purchased from VWR International, LLC (Radnor, PA, USA).

FACS analyses were collected on Attune NxT or BD LSR Fortessa flow cytometers and analyzed on FlowJo v7.6 and FCS Express v6 and v7. Fluorescent images were taken with a EVOS microscope (Thermofisher, Waltham, MA, USA). Confocal laser scanning microscopy (CLSM) images were taken by using a Zeiss LSM 700 Confocal Microscope (Carl Zeiss, Thornwood, NY, USA). High-performance liquid chromatography (HPLC) analysis was performed on a Shimadzu CBM-20A system (Shimadzu, Kyoto, Japan) equipped with an SPD-20A PDA detector (190-800 nm), an RF-20A fluorescence detector, and an analytical C18 column (Shimadzu, 3 μm, 50×4.6 mm, Kyoto, Japan). Nuclear Magnetic Resonance (NMR) spectra were recorded on a Varian U500 (500 MHz) or VXR500 (500 MHz), or a Bruker Carver B500 (500 MHz) spectrometer. Electrospray ionization (ESI) mass spectra were obtained from a Waters ZMD Quadrupole Instrument (Waters, Milford, MA, USA). Matrix-assisted laser desorption/ionization (MALDI) spectra were collected on the Bruker Ultraflextreme MALDI-TOF/TOF Mass Spectrometer. In vivo and ex vivo images of animals and tissues were taken on a Bruker In Vivo Imaging System (Bruker, Billerica, MA, USA). Preparative HPLC was performed on a CombiFlash®Rf system (Teledyne ISCO, Lincoln, NE, USA) equipped with a RediSep®Rf HP C18 column (Teledyne ISCO, 30 g, Lincoln, NE, USA). Lyophilization was conducted in a Labconco FreeZone lyophilizer (Kansas City, MO, USA). Magnetic resonance imaging (MRI) scans were performed on a 9.4 T Bruker 30 cm AVANCE NEO equipped with a dual channel transmit, 4 receiver channels, a 4-channel mouse brain array, and a 4-channel rat brain array coil.

Cell lines and animals. The MEL cell line was a generous gift from Dr. Martin Burke's lab at the University of Illinois at Urbana-Champaign. Cells were cultured in DMEM containing 10% FBS, 100 units/mL Penicillin G, non-essential amino acids, and 5% BSA at 37° C. in 5% CO₂ humidified air. Female C57BL/6 and Balb/c mice were purchased from the Jackson Laboratory (Bar Harbor, ME, USA). Feed and water were available ad libitum. Artificial light was provided in a 12 h/12 h cycle. All procedures involving animals were done in compliance with National Institutes of Health and Institutional guidelines with approval from the Institutional Animal Care and Use Committee (IACUC) at the University of Illinois at Urbana-Champaign.

Synthesis of AAM, AAGal and AAGlu. Mannosamine hydrochloride or galactosamine hydrochloride or glucosamine hydrochloride (1.0 e.q.) was dissolved in anhydrous methanol and cooled to 0° C., followed by the addition of sodium methoxide in methanol (1.0 e.q.). Chloroacetic anhydride (1.05 e.q.) and triethylamine (1.0 e.q.) were then added. The reaction mixture was stirred at room temperature for overnight. Upon removal of the solvent, the crude product was re-dissolved in water. Sodium azide (4.0 e.q.) was then added and the reaction mixture was stirred at 60° C. for overnight. After removing the solvent, the crude product was re-dissolved in pyridine, followed by the addition of acetic anhydride and 4-dimethylaminopyridine. After 24 h, the solvent was removed and the crude product was purified via silica column chromatography using ethyl acetate and hexane as the eluents. ¹H NMR of AAM (CDCl₃, 500 MHz): δ (ppm) 6.66&6.60 (d, J=9.0 Hz, 1H, C(O)NHCH), 6.04&6.04 (d, 1H, J=1.9 Hz, NHCHCHO), 5.32-5.35&5.04-5.07 (dd, J=10.2, 4.2 Hz, 1H, CH₂CHCHCH), 5.22&5.16 (t, J=9.9 Hz, 1H, CH₂CHCHCH), 4.60-4.63&4.71-4.74 (m, 1H, NHCHCHO), 4.10-4.27 (m, 2H, CH₂CHCHCH), 4.07 (m, 2H, C(O)CH₂N₃), 3.80-4.04 (m, 1H, CH₂CHCHCH), 2.00-2.18 (s, 12H, CH₃C(O)). ¹³C NMR (CDCl₃, 500 MHz): δ (ppm) 170.7, 170.4, 170.3, 169.8, 168.6, 168.3, 167.5, 166.9, 91.5, 90.5, 73.6, 71.7, 70.5, 69.1, 65.3, 65.1, 62.0, 61.9, 52.8, 52.6, 49.9, 49.5, 21.1, 21.0, 21.0, 20.9, 20.9, 20.9, 20.8. ESI MS (m/z): calculated for C₁₆H₂₂N₄O₁₀Na [M+Na]⁺ 453.1, found 453.1.

In vitro metabolic labeling of MEL cells. MEL cells were cultured in DMEM containing non-essential amino acids, 10% FBS, and 5% BSA at the density of 5×10⁵/mL for 12 h. AAM or AAGal or AAGlu with a final concentration of 50 or 100 μM was added to the culture medium. At selected time points, cells were collected, washed with opti-MEM twice, and incubated with DBCO-Cy5 (10 μg/mL) and SYTO™ 85 Orange Fluorescent Nucleic Acid Stain (5 μM) at room temperature for 30 min. After washing, cells were analyzed on a flow cytometer. To initiate the differentiation of MEL cells, 2% DMSO and 50 μM iron citrate were added. The counts and size of cells were analyzed via a cell-counter, and hemoglobin was detected by analyzing UV absorption at 438 nm.

In vitro metabolic labeling of primary RBCs. Blood was collected from C57BL/6 or Balb/c mice, and RBCs were harvested after the removal of lymphocytes. To maintain the viability of mouse RBCs in vitro, DMEM media containing non-essential amino acids, 10% FBS, 5% BSA, and 10 g/L glucose were used. RBCs were seeded at 10⁸/mL in 24-well plates in the presence of 50 or 100 μM AAM or AAGal. After 24 h, 10⁷ RBCs were taken out, washed with Alsever's solution twice, and incubated with DBCO-Cy5 (10 μg/ml) and SYTO™ 85 Orange Fluorescent Nucleic Acid Stain (5 μM) at room temperature for 1 h. Cells were washed and analyzed on a flow cytometer.

In vivo metabolic labeling of RBCs. C57BL/6 or Balb/c mice were intravenously injected with AAM (100 mg/kg) or PBS twice daily for 3 consecutive days. At 1, 3, 5, 7, 14, 21, 28, 35, 42, and 49 days post the last injection of AAM or PBS, blood was collected and stored in Alsever's solution. After washing, cells were stained with DBCO-Cy5 (5 μg/mL) and SYTO™ 85 Orange Fluorescent Nucleic Acid Stain (5 μM) at room temperature for 1 h. After washing with Alsever's solution for three times, samples were run on a flow cytometer. For some experiments, AAM (200 mg/kg) was i.p. injected for 2, 4, and 6 times with an interval of 12 h.

For the analysis of RBC precursor cells in the bone marrow, at 48 h post the last injection of AAM, bone marrow cells in the tibia and femur of mice were collected and stained with DBCO-Cy5, anti-ter119, anti-CD44, and fixable viability dye, prior to flow cytometry analysis. To detect azido-labeled cells in healthy tissues, tissues were sectioned with a thickness of 20 μm, stained with DBCO-Cy5 and DAPI for 20 min, washed with PBS for multiple times, and mounted onto a microscope slide with the addition of Prolong Gold, prior to confocal imaging.

Enrichment of azido-labeled RBCs. At 7 days post AAM injection, RBCs were harvested and incubated with DBCO-desthiobiotin (10 μg/mL) for 1 h at room temperature. Cells were then incubated with streptavidin-coated magnetic microbeads and separated using a MACS column. Azido-labeled cells were eluted with biotin-containing buffer and analyzed by flow cytometry and Western blot.

Fluorescence imaging of RBCs. Blood was drawn from mice at selected times post the injection of AAM or PBS, and kept in the Alsever's solution. After washing, RBCs were incubated with 5 μg/mL DBCO-Cy5 at room temperature for 1 h, washed with Alsever's solution for 5 times, and added to a glass coverslip. Samples were stored at 4° C. prior to fluorescence imaging.

RBC agglutination analysis. C57BL/6 mice were intravenously injected with AAM (100 mg/kg) or PBS twice daily for 3 days. At different times after the final AAM injection, blood was extracted from the tail of mice and diluted with the Alsever's solution. A fraction of cells were directly imaged under a fluorescence microscope for morphology and agglutination analysis. Cells were also analyzed on a flow cytometer. The singlets were quantified from the FSC-A/FSC-H plot.

ATP level measurement. C57BL/6 mice were intravenously injected with AAM (100 mg/kg) or PBS twice a day for 3 days. At different times after the final AAM injection, blood was extracted from the tail of mice and washed with Alsever's solution twice. The cell pellet was suspended in ATP-free water, followed by three freeze-thaw cycles to lyse RBCs. The ATP level in RBC lysates was measured using the ENLITEN® ATP Assay System Bioluminescence Detection Kit.

Resorufin assay of RBCs. C57BL/6 mice were intravenously injected with AAM (100 mg/kg) or PBS twice a day for 3 days. At different times after the final AAM injection, blood was extracted from the tail of mice and washed with Alsever's solution twice. The cell pellet was resuspended in Alsever's solution and incubated with resazurin (50 μg/mL) at 37° C. for 15 min and 2 h, respectively. The resorufin product was detected on a plate reader (λex/λem=560/590 nm). RBCs incubated with H₂O₂ at room temperature for 10 min were used as the negative control.

In vivo targeting of RBCs. C57BL/6 mice were intravenously injected with AAM (100 mg/kg) or PBS twice a day for 3 days. At 3 days post the last injection of AAM, DBCO-Cy5 (5 mg/kg) was intravenously injected. At selected times, blood was collected and placed in the Alsever's solution. After washing, cells were incubated with SYTO™ 85 Orange Fluorescent Nucleic Acid Stain (5 μM) at room temperature for 30 min, washed, and analyzed on a flow cytometer.

Western blot analysis. C57BL/6 mice were intravenously injected with AAM (100 mg/kg) or PBS twice daily for 3 days. Blood was collected at 7 days post the final injection of AAM or PBS. White blood cells were removed with Histopaque®-1077 according to the manufacturer's protocol. RBCs were lysed with 0.1×PBS containing Halt™ Protease Inhibitor Cocktail at 4° C. for 30 min, followed by centrifugation at 21,000 g for 30 min and washing with 0.1×PBS containing Halt™ Protease Inhibitor Cocktail for 5 times to yield the RBC membranes. RBC membranes were lysed and incubated with alkyne-PEG₄-biotin for overnight at 4° C. After washing with 0.1×PBS, proteins were loaded into SDS-PAGE gels for electrophoresis and subsequent transfer to the nitrocellulose membrane. The nitrocellulose membrane was blocked with the blocking buffer (5% non-fat milk in PBST) for overnight at 4° C., incubated with streptavidin-HRP for 30 min, washed with PBST for 5 times, and treated with HRP substrate for chemiluminescence imaging on an ImageQuant 800. Protein bands were also stained with Ponceau S.

RBC lipid extraction and analysis. C57BL/6 mice were intravenously injected with AAM (100 mg/kg) or PBS twice daily for 3 days. Blood was collected at 7 days post the final injection of AAM or PBS. After removing the white blood cells with Histopaque®-1077, RBCs were incubated with DBCO-Cy5 (10 μg/mL) in Alsever's solution for 1 h at room temperature. Cells were then washed with Alsever's solution for three times and resuspended in a mixture of methanol and dichloromethane (3/7, v/v). The samples were then sonicated for 10 min. The protein precipitate was removed by centrifugation at 21,000 g for 10 min at 4° C. The lipid-containing supernatant was gently collected and analyzed on an HPLC.

Ex vivo retention of Cy5 on RBC surface. RBCs were isolated from mice at 7 days post injections of AAM or PBS, and incubated with DBCO-Cy5 (5 μg/mL) for 1 h at room temperature. Cells were washed three times and resuspended in Alsever's solution. An aliquot of sample was directly analyzed on the flow cytometer, and the remaining sample was stored at 4° C. At selected times, an aliquot of each sample was run on the flow cytometer.

In vivo fluorescence imaging of blood vessels and tumors. For B16F10 tumor study, C57BL/6 mice were intravenously injected with AAM (100 mg/kg) or PBS twice a day for 3 days. DBCO-Cy5 (5 mg/kg) was intravenously injected at 4 days post the final injection of AAM or PBS. Blood was drawn from the tail at selected times for fluorescence imaging. At 17 days post DBCO-Cy5 injection, 5×10⁶ B16F10 cells were subcutaneously injected into the flank of C57BL/6 mice. Tumors were collected 7 days after inoculation and subjected to IVIS imaging. For 4T1 tumor study, Balb/c mice were intravenously injected with AAM (100 mg/kg) or PBS twice daily for 3 days. At 4 days post the final injection of AAM or PBS, 2×10⁶ 4T1 cells were subcutaneously injected into the flank of mice. After 3 days, DBCO-Cy5 (5 mg/kg) was i.p. injected. Blood was drawn at selected times for fluorescence imaging and flow cytometry analysis. IVIS imaging of mice was also performed at selected times. At 21 days post tumor cell inoculation, tumors and organs were harvested for IVIS imaging. During the 4T1 tumor imaging study, at selected times, blood was also collected for the analysis of Cy5 signal in the plasma. The blood cells were removed by centrifugation at 2,000 g for 10 min, and the supernatant was harvested for Cy5 fluorescence measurement on a plate reader.

Synthesis of DBCO-DOTA-Gd. 1,4,7,10-Tetraazacyclododecane-1,4,7,10-tetraacetic acid (DOTA, 1.1 e.q.) and DBCO-PEG₄-amine (1.0 e.q.) were dissolved in dimethyl sulfoxide, followed by the addition of N,N-diisopropylethylamine (DIPEA) (4.4 e.q.) and hexafluorophosphate azabenzotriazole tetramethyl uronium (1.0 e.q.). After 2 h, the solvent was removed and the residue was purified by preparative HPLC to yield DBCO-DOTA. DBCO-DOTA (1.0 e.q) and DIPEA (3.5 e.q.) were then dissolved in water, followed by the addition of Gd(NO₃)₃·6H₂O (1.5 e.q.). After 4 h, the solvent was removed and the residue was purified by preparative HPLC to yield DBCO-DOTA-Gd.

MRI of mice. C57BL/6 mice were intravenously injected with AAM (100 mg/kg) or PBS twice a day for 3 days. At 4 days post the final injection of AAM or PBS, DBCO-DOTA-Gd (250 mg/kg) was intravenously injected. Mice were imaged before the injection and at different times after in a 9.4 T Bruker system (Bruker BioSpec 94/30 USR) using a 1H receive-only 2×2 mouse brain surface array coil. The mice were initially anesthetized with 2.5% isoflurane and maintained at 0.5-1.5% isoflurane throughout the experiment. Body temperature was held at 37° C. by circulating warm water and monitored using a rectal thermosensor. The Small Animal Monitoring & Gating System (SA Instruments) was utilized for continuous monitoring of both respiration rate and body temperature, ensuring the respiration rate was controlled within the range of 35 to 65 per minute. For contrast-enhanced magnetic resonance angiography (CE-MRA) to compare blood vessel visualization, a 3D T1-weighted FLASH sequence was used with coronal slices to minimize tight of flight effects. Scan parameters were: TE=2.11 ms, TR=15 ms, readout bandwidth=59 kHz, number of averages=4, flip angle=15, FOV=23.25×14.23×13.27 mm³, matrix size=200×180×72, and total scan time=10 min. The same parameters were used for the scans.

DBCO-doxorubicin synthesis. DBCO-NHS and DIPEA were dissolved in acetonitrile, followed by the addition of hydrazine monohydrate. The mixture was stirred for 30 min. After removing the solvent, the crude product was purified via preparative HPLC to yield DBCO-hydrazide. DBCO-hydrazide and doxorubicin were then dissolved in anhydrous methanol, followed by the addition of trifluoroacetic acid. After the complete consumption of DBCO-hydrazide, the solvent was removed under reduced pressure. The crude product was purified via preparative HPLC to yield DBCO-doxorubicin.

DBCO-doxorubicin degradation. DBCO-doxorubicin was dissolved in buffers with a pH of 7.4, 6.5, and 5.5, respectively and incubated at 37° C. At selected times, 10 μL aliquots were run on an HPLC to determine the degradation kinetics of DBCO-doxorubicin.

In vivo targeting of DBCO-doxorubicin to RBCs. C57BL/6 mice were intravenously injected with AAM (100 mg/kg) or PBS twice a day for 3 days. At 3 days post the final injection of AAM or PBS, DBCO-doxorubicin (5 mg/kg) in a mixture of DMSO/PBS (1/9, v/v) was i.v injected. At 2 or 24 h post the injection of DBCO-doxorubicin, blood was collected from mice and stored in Alsever's solution. After staining with SYTO™ 85 Orange Fluorescent Nucleic Acid Stain, cells were analyzed on a flow cytometer.

DBCO-PE synthesis and conjugation to RBC. PE (1.0 e.q.) was dissolved in PBS, followed by the addition of DBCO-PEG₄-NHS (10 e.q.). The mixture was stirred at 4° C. for overnight. After removing small molecules using the Sephadex G-25 resin column, the purified DBCO-PE was concentrated via ultracentrifugation with a 100 kDa ultra centrifugal filter. RBCs isolated from AAM- or PBS-treated mice were incubated with DBCO-PE in Alsever's solution for 1 h at room temperature. After washing with Alsever's solution for three times, cells were stained with a DNA dye and analyzed on a flow cytometer.

Synthesis of DBCO-insulin. DBCO-ester-NHS was first synthesized. DBCO-PEG₃-OH (1.0 e.q.) and succinic anhydride (2.0 e.q.) were dissolved in anhydrous pyridine, followed by the addition of 4-dimethylaminopyridine (1.0 e.q.). The mixture was stirred for overnight. After removing the solvent, the crude product was purified using preparative HPLC to yield DBCO-ester-COOH. DBCO-ester-COOH (1.0 e.q.) was then dissolved in dichloromethane, followed by the addition of 1-ethyl-3-(3-dimethylaminopropyl)carbodiimide (1.2 e.q.) and N-hydroxysuccinimide (2.0 e.q.). The mixture was stirred overnight. After removing the solvent, the crude product was purified via silica gel column chromatography to yield DBCO-ester-NHS. The as-synthesized DBCO-ester-NHS (2.0 e.q.) and insulin (1.0 e.q.) were dissolved in PBS and stirred for overnight at 4° C. The crude product was purified via ultracentrifugation using a 3 k Da ultracentrifugal filter.

In vitro conjugation of DBCO-insulin to cells. 5×10⁴ 4T1 cells in 96-well plates were incubated with AAM (50 μM) or PBS for 24 h. Cells were detached with trypsin and incubated with DBCO-insulin (0.1 mg/mL) in FACS buffer (2% BSA in PBS) for 1 h at 4° C. After washing, cells were stained with rabbit anti-insulin primary antibody for 30 min at 4° C., and then incubated with Cy5-conjugated goat anti-rabbit secondary antibody for 30 min, prior to flow cytometry analysis. Similarly, RBCs isolated from AAM- or PBS-treated mice were incubated with DBCO-insulin in Alsever's solution (0.1 mg/mL) for 1 h, followed by the staining with rabbit anti-insulin primary antibody and Cy5-conjugated goat anti-rabbit secondary antibody. Cells were also stained with a DNA dye. After washing, RBCs were analyzed on a flow cytometer.

STZ induced type 1 diabetes (TID). To induce TID, female C57BL/6 mice were injected with STZ (150 mg/kg). After monitoring the blood glucose levels for 7 days and confirming the onset of T1D (blood glucose level >220 mg/dL without fasting), mice were randomly divided into 4 groups. Mice were intravenously injected with AAM (100 mg/kg) or PBS twice daily for 3 days. Four days later, DBCO-insulin or insulin (10 IU/kg) was i.p injected. For the measurement of plasma insulin levels, blood was collected at 8 h post injection of DBCO-insulin or insulin and placed in heparin-coated tubes. After removing blood cells via centrifugation, the supernatant was harvested for the quantification of insulin using the insulin ELISA kit (Catalog 80-INSHU-E01.1).

Glucose tolerance test (GTT). Mice were fasted for 12 h, and DBCO-insulin or insulin (10 IU/kg) was i.p injected (counted as time 0). At selected times, blood was drawn from the tail, and the glucose level in the blood was measured using the Clarity BG1000 Blood Glucose Meter and Strips. At 3 h and 6 h, 1.5 g/kg glucose was i.p injected to each mouse. The blood glucose level was continuously monitored for 9 h with an interval of 0.5 h. After the GTT experiment, DBCO-insulin or insulin was injected every week for 2 more injections (3 injections in total). The body weight of mice was closely monitored.

Statistical analyses. Statistical analysis was performed using GraphPad Prism v6 and v8. Sample variance was tested using the F test. For samples with equal variance, the significance between the groups was analyzed by a two-tailed student's t test. For samples with unequal variance, a two-tailed Welch's t-test was performed. For multiple comparisons, a one-way analysis of variance (ANOVA) with post hoc Fisher's LSD test was used. The results were deemed significant at 0.01<*P≤0.05, highly significant at 0.001<**P≤0.01, and extremely significant at ***P≤0.001.

Statistics & Reproducibility. The sample sizes were determined empirically. In general, in vitro studies involve n=3-6 and in vivo studies involve n=3-7. No data were excluded from the analyses. Randomization was used for animal studies.

### Example 2

### In Vitro Metabolic Labeling of Red Blood Cells

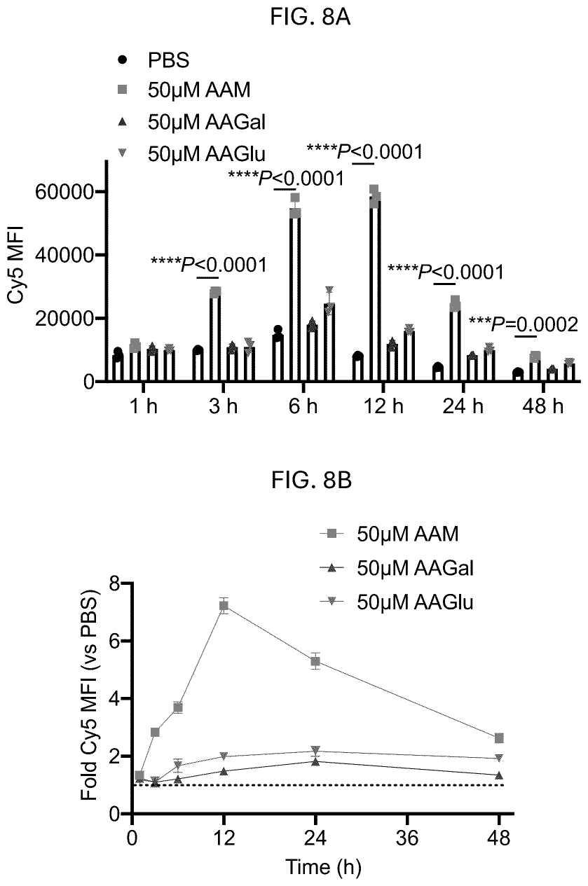

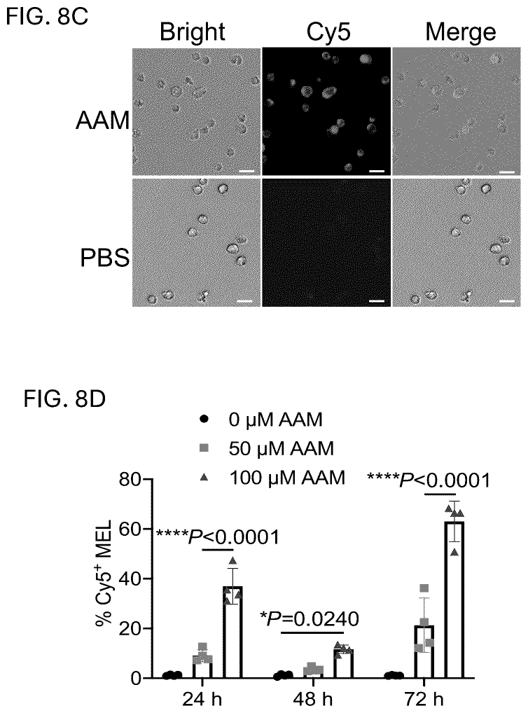

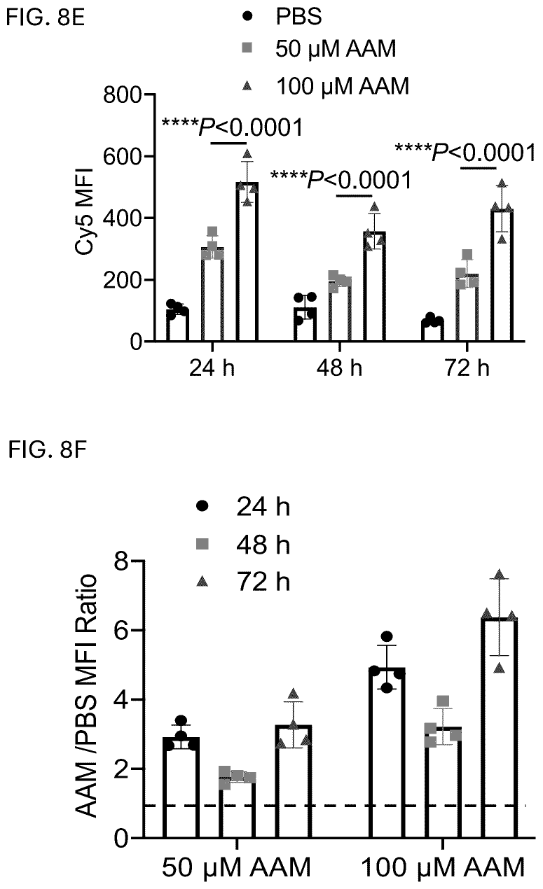

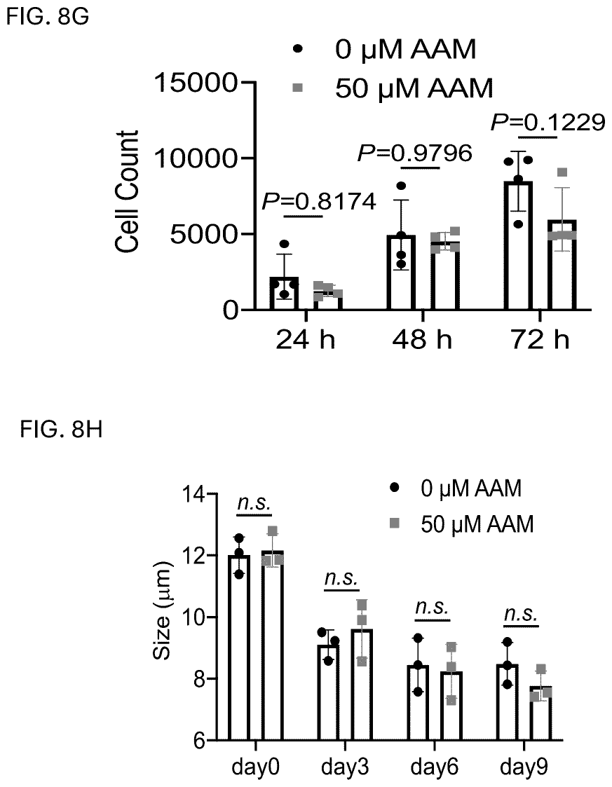

We first screened the metabolic labeling efficiency of three types of azido-sugars, AAM, tetraacetyl-N-azidoacetylgalactosamine (AAGal), and tetraacetyl-N-azidoacetylglucosamine (AAGlu) using murine erythroleukemia (MEL) cells, a precursor cell of RBCs. MEL cells were incubated with 50 μM AAM, AAGal, or AAGlu for varied times, followed by the detection of cell-surface azido groups using DBCO-Cy5. Compared to control cells without azido-sugar treatment, cells treated with AAM, AAGal, or AAGlu showed significantly higher Cy5 fluorescence intensity (FIGS. 8A-8C), demonstrating the successful labeling of MEL cells with azido groups. AAM exhibited a higher labeling efficiency than AAGal and AAGlu (FIGS. 8A-8B). As MEL cells can proliferate rapidly, the amount of cell-surface azido groups decreased from 24 h to 48 h (FIGS. 8D-8F). By adding fresh AAM to the media at 48 h, the density of cell-surface azido groups could be further increased (FIGS. 8D-8F). It is noteworthy that AAM treatment induced negligible changes in the viability and differentiation of MEL cells (FIGS. 8G-8H).

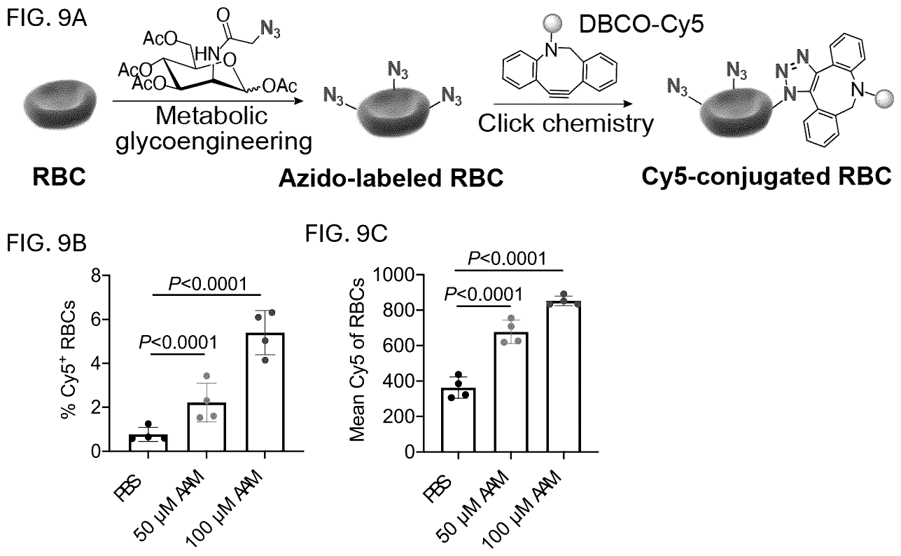

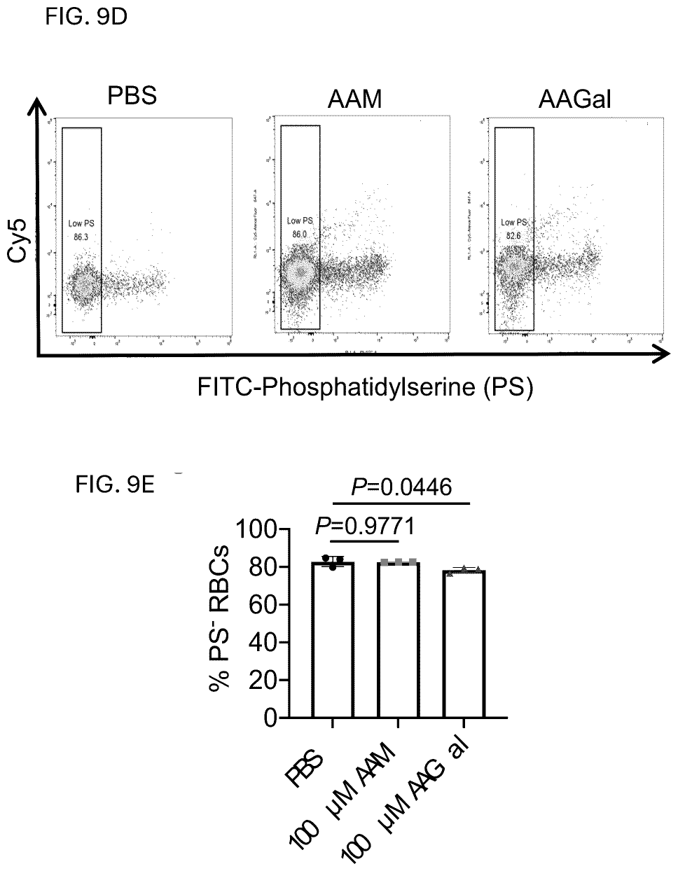

We next studied whether AAM can metabolically label primary RBCs in vitro (FIG. 9A). As conventional Alsever's solution and FBS-containing DMEM media failed to maintain the long-term survival of primary RBCs, we tested different combinations of medium components and narrowed down to a type of DMEM medium containing 10% FBS, 5% BSA, non-essential amino acids, and 10 g/L glucose, which was able to maintain the high viability of RBCs isolated from C57BL/6 mice for up to 120 h in vitro. Mouse RBCs were incubated with AAM for 24 h, and the cell-surface azido groups were detected by DBCO-Cy5. Compared to control cells, AAM-treated RBCs showed significantly higher Cy5 fluorescence intensity (FIGS. 9B-9C), indicating the successful labeling of RBCs with azido groups. We also studied whether AAM treatment could induce the apoptosis of RBCs by examining the expression level of phosphatidylserine, which showed negligible differences between AAM- and PBS-treated RBCs (FIGS. 9D-9E). These experiments demonstrated that AAM can metabolically label primary RBCs with azido groups in vitro, without inducing apoptosis or death of RBCs.

### Example 3

### In Vivo Metabolic Labeling of Circulating Red Blood Cells

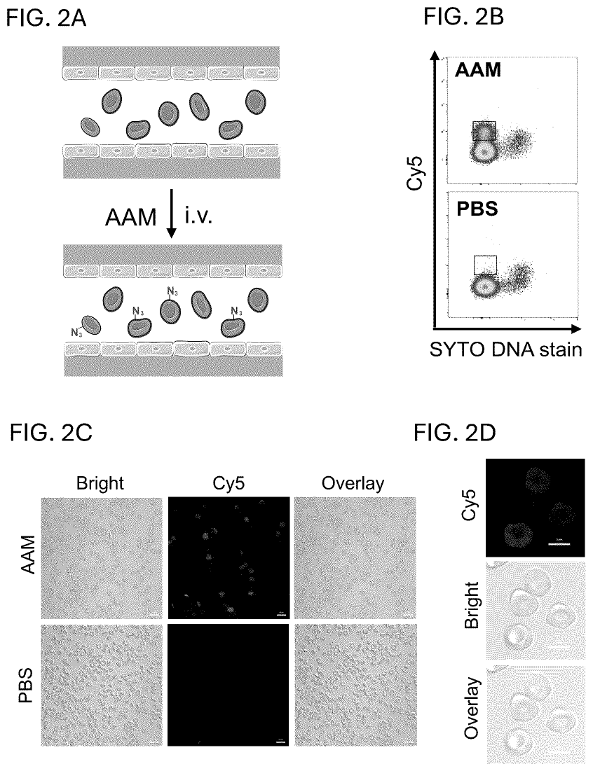

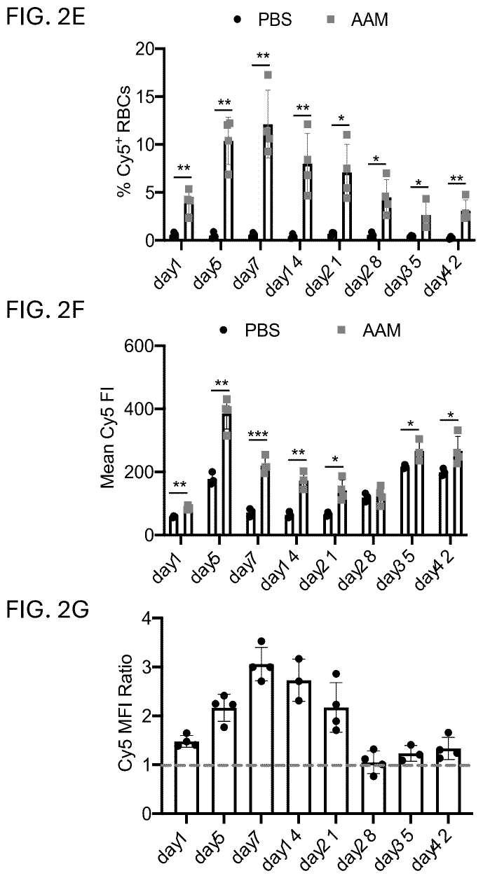

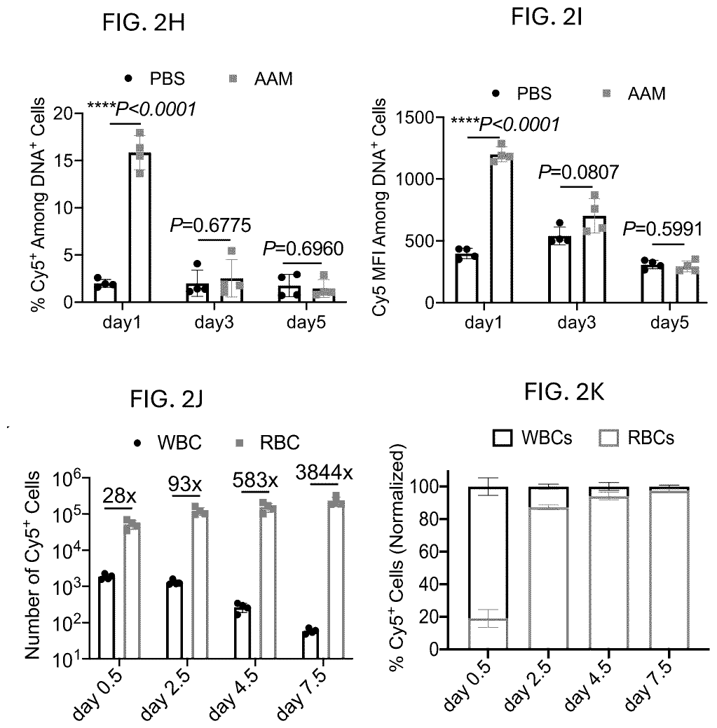

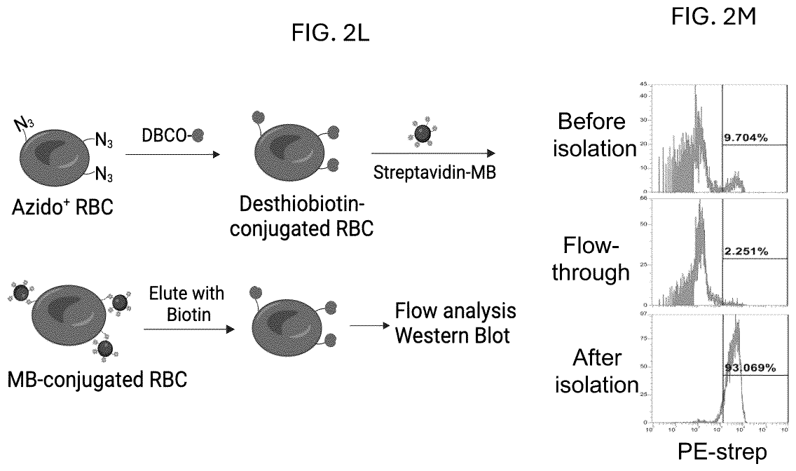

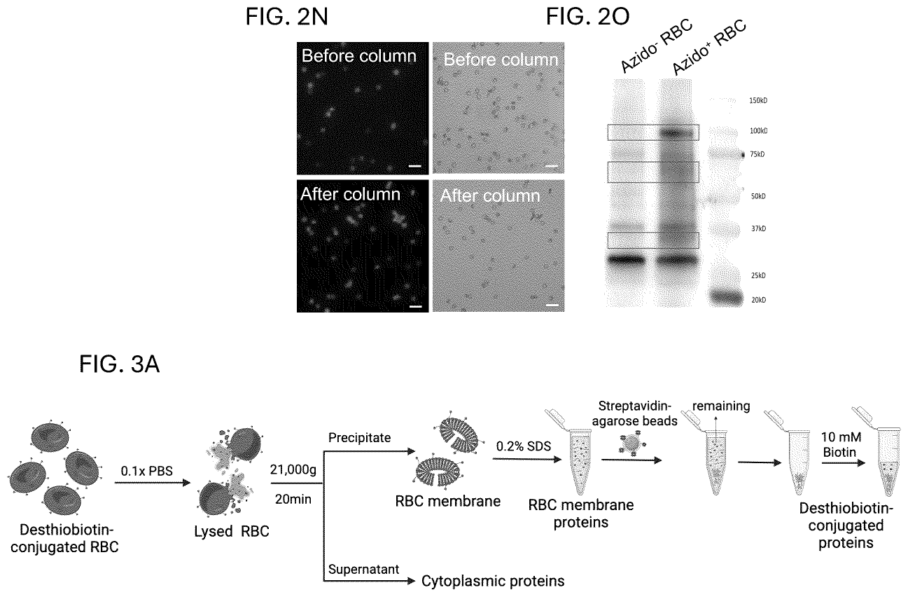

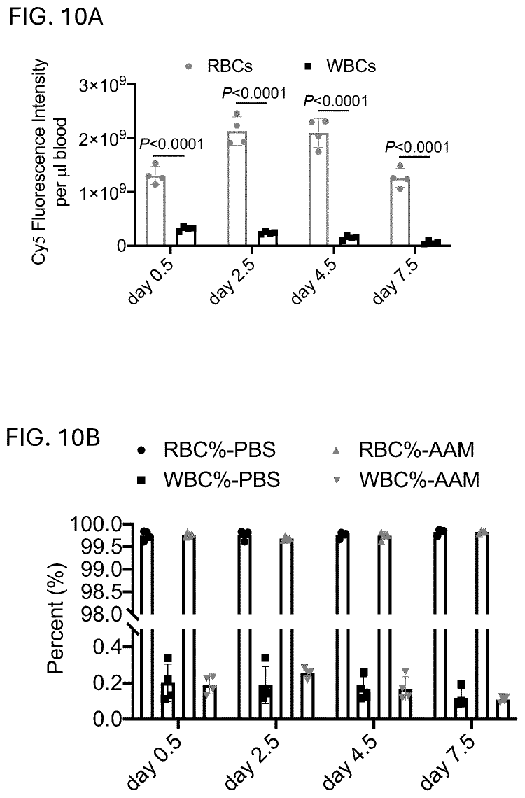

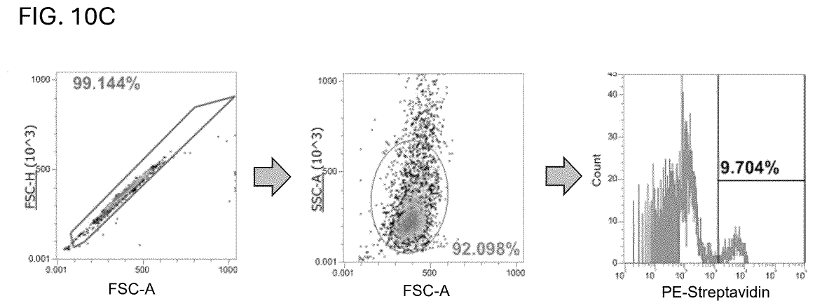

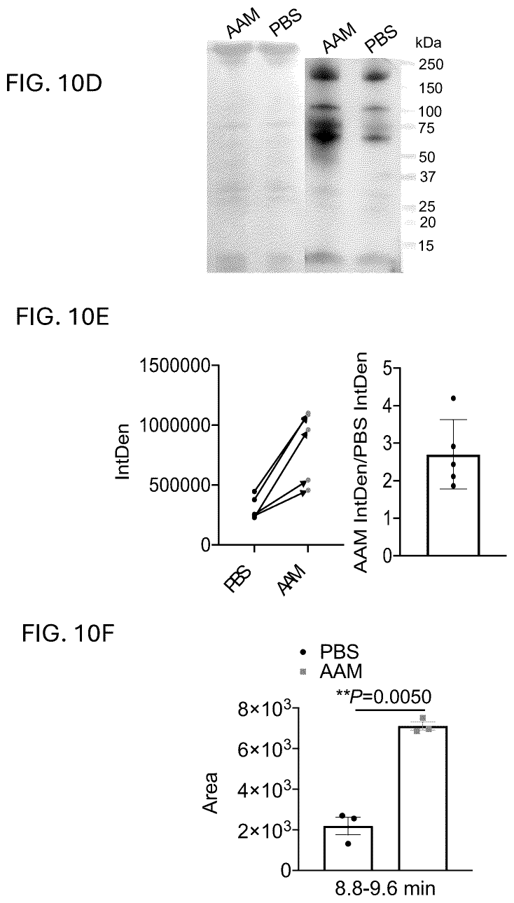

We next studied whether systemically administered AAM can metabolically label RBCs with azido groups in vivo (FIG. 2A). C57BL/6 mice were intravenously administered AAM (100 mg/kg) twice daily for three days, followed by the collection of RBCs at different times for azido detection via DBCO-Cy5. Compared to control RBCs that showed minimal Cy5 signal, RBCs collected from AAM-treated mice at 14 days post AAM injections showed a distinct Cy5⁺ population in FACS assay (FIG. 2B), indicating the successful metabolic labeling of RBCs with azido groups. Fluorescence imaging also confirmed the much higher Cy5 fluorescence intensity of RBCs from AAM-treated mice (FIGS. 2C-2D). The percentage of azide⁺ RBCs increased from 4.2% at 24 h to 10.4% at 5 days, and further to 11.3% at 7 days (FIG. 2E). While the percentage of azide⁺ RBCs started to decrease from day 7, a notable fraction of azide⁺ RBCs could be consistently detected for over 42 days (FIGS. 2E-2F), nearly the lifespan of mouse RBCs (˜45 days). The Cy5 fluorescence intensity ratio of RBCs (AAM/PBS) constantly stayed above 1.0, with a maximal ratio of 3.1 at 7 days (FIG. 2G). These experiments demonstrated the successful metabolic labeling of RBCs with azido groups in vivo and the superior retention of azido groups on the surface of RBCs. It was not surprising to detect azido-labeled immune cells and other cells in the bloodstream at 24 h post the injections of AAM (FIGS. 2H-2I). However, due to their high proliferation rate and active metabolism, the number of azido groups decayed to the baseline level after 3 days (FIGS. 2H-2I). The number of azide+ RBCs was 28-fold, 93-fold, 583-fold, and 3,844-fold greater than azido+ white blood cells (WBCs) at 0.5, 2.5, 4.5, and 7.5 days post the injections of AAM (FIGS. 2J-2K, FIG. 10A). It is noteworthy that AAM treatment did not alter the percentage and number of RBCs and WBCs in the bloodstream (FIG. 10B).

To further confirm the successful metabolic labeling of RBCs, we performed magnetic selection of azido-positive RBCs collected from C57BL/6 mice at 7 days post injections of AAM via DBCO-desthiobiotin conjugation, streptavidin-microbead pull-down, and biotin elution (FIG. 2L). Flow cytometry analyses confirmed the successful enrichment of desthiobiotin-tagged RBCs (FIG. 2M-2N, FIG. 10C). Western blot analysis of the enriched azido/desthiobiotin-tagged RBCs versus control azido-RBCs was able to identify several types of azido-labeled proteins such as band 3 proteins (100 kDa), bands 4 and 5 proteins (55-60 kDa), and glycophorins A-D (30-35 kDa) (FIG. 2O). It is noteworthy that Western blot analysis of RBCs, without magnetic enrichment, also showed a difference between AAM and PBS groups (FIGS. 10D-10E).

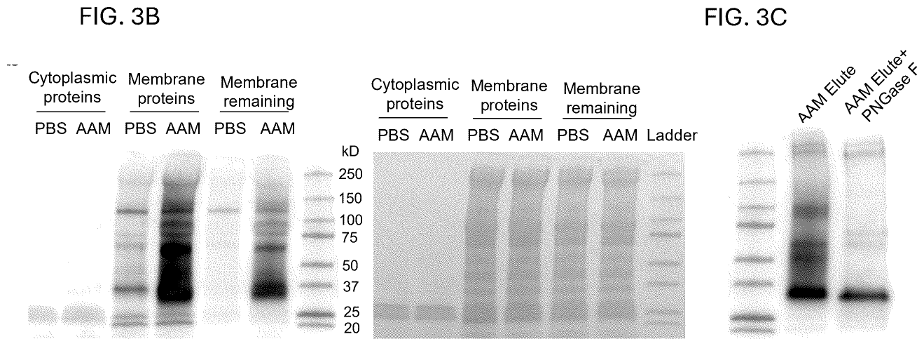

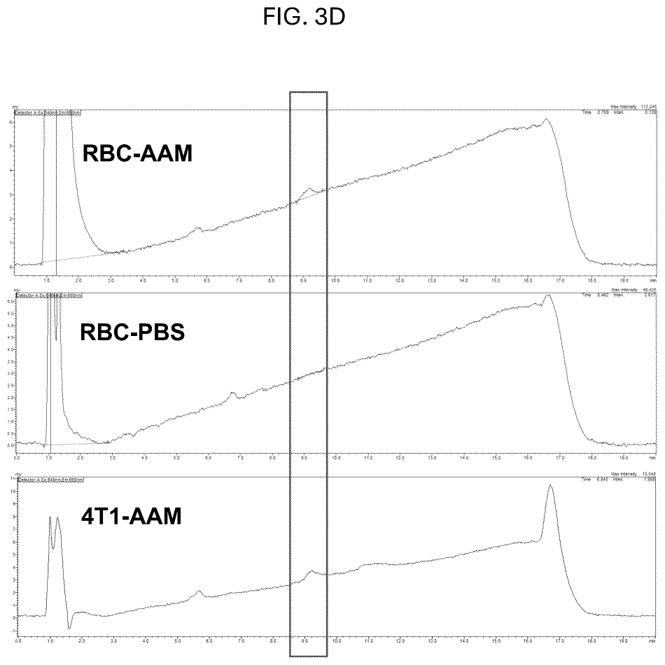

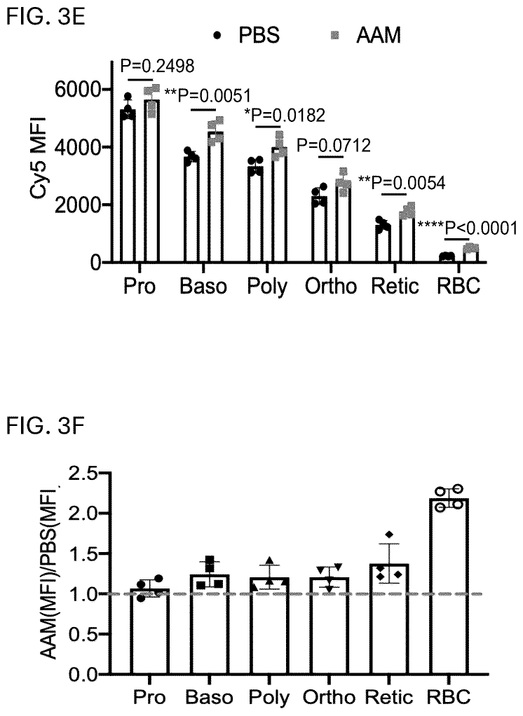

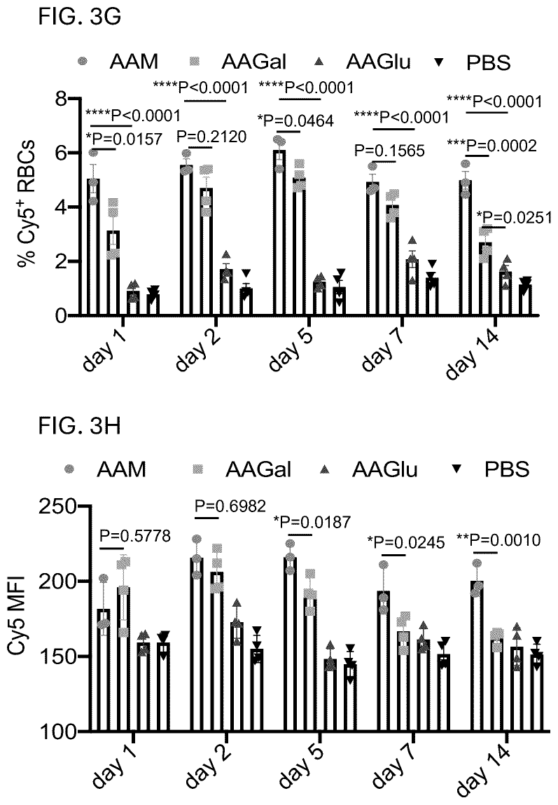

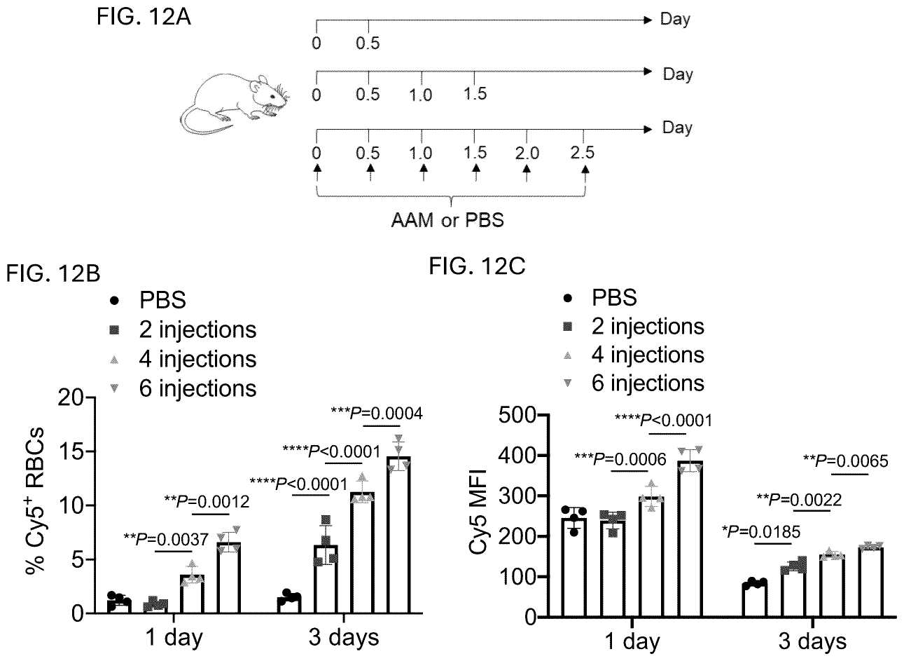

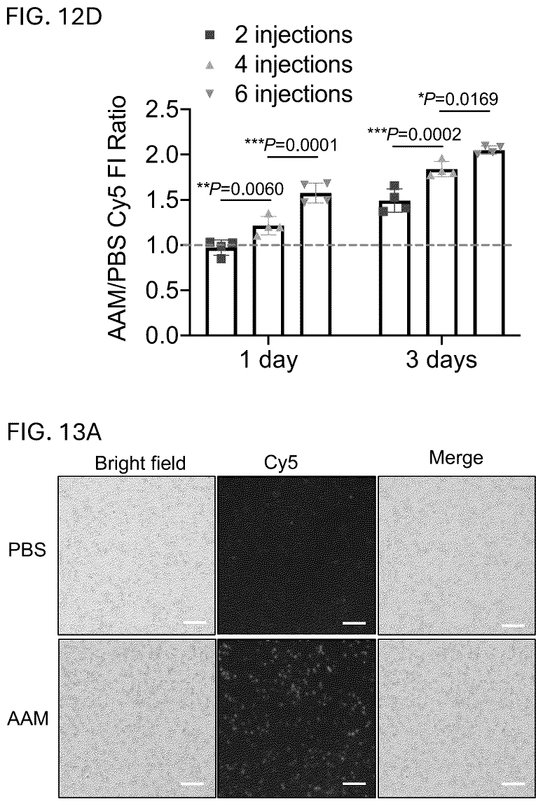

In vivo RBC labeling efficiency of AAM, AAGal, and AAGlu were assessed. C57BL/6 mice were intravenously injected with AAM, AAGal, AAGlu, or PBS twice daily for three days. RBCs were collected at multiple time points post-injection, and stained with DBCO-Cy5 for flow cytometry analysis. Compared to AAGlu, both AAM and AAGal showed a significantly higher RBC labeling efficiency (FIGS. 3G-3H). Compared to the AAGal group, azido-labeled RBCs in AAM-treated mice exhibited a higher stability over time (FIGS. 3G-3H). In addition to intravenous injections, intraperitoneal injections of AAM were tested on labeling of RBCs with azido groups. In addition to intravenous injections, intraperitoneal injections of AAM, which allowed for the use of a higher dose, also resulted in successful metabolic labeling of RBCs with azido groups (FIGS. 12A-12D). The RBC metabolic labeling efficiency increased with the dosing frequency of AAM, with up to 14.5% of azido-labeled RBCs among all the circulating RBCs (FIGS. 12A-12D).

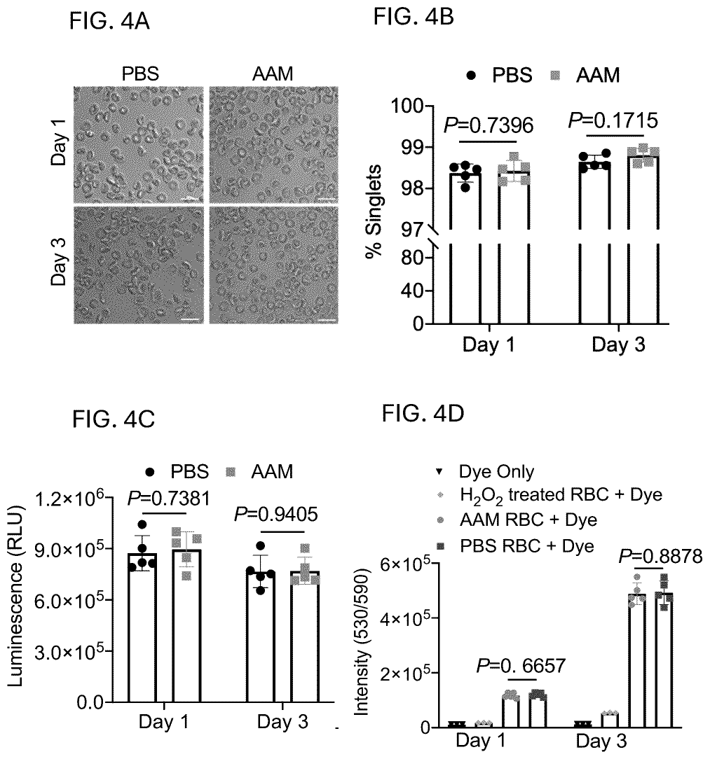

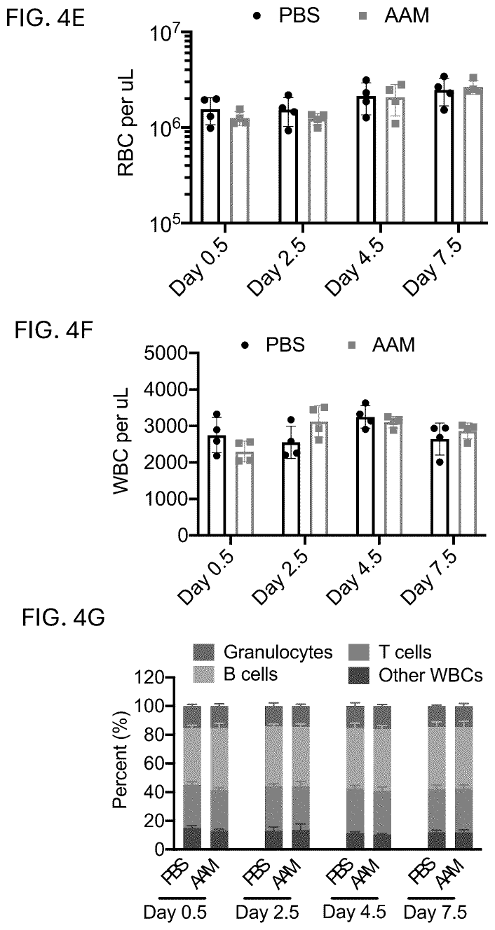

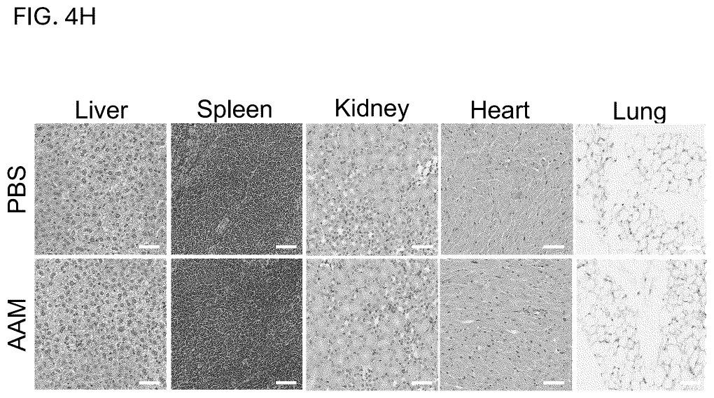

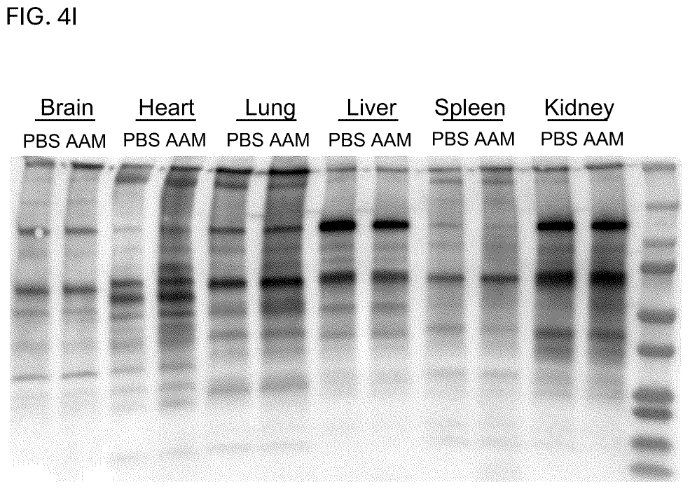

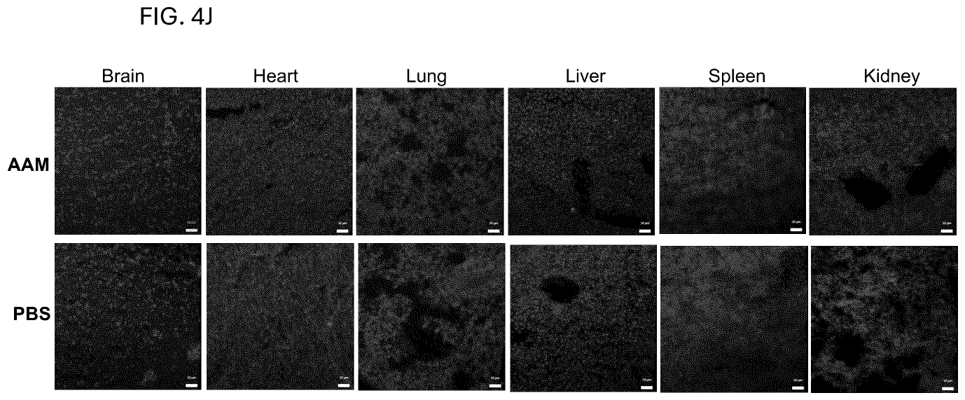

We also studied whether in vivo metabolic labeling of RBCs could pose any toxic effect on RBCs and tissues. Following intravenous injections of AAM or PBS into C57BL/6 mice twice daily for three days, RBCs were isolated at 1 and 3 days, respectively. RBCs from AAM- or PBS-treated mice both showed the normal biconcave disc morphology and exhibited minimal agglutination (FIG. 4A), which was further confirmed by flow cytometry (FIG. 4B). As a key indicator of RBC metabolic states, the intracellular ATP level was measured and compared between AAM and PBS groups. Per the ATP bioluminescence assay, RBCs from AAM- or PBS-treated mice showed negligible differences in intracellular ATP levels (FIG. 4C). In addition to ATP, we also measured the intracellular NAD(H)/NADP(H) levels of RBCs using the resazurin assay. Compared to control RBCs, RBCs in AAM-treated mice showed negligible changes in NAD(H)/NADP(H) levels, both showing a much higher NAD(H)/NADP(H) level than the negative control (FIG. 4D).

To study the potential chronic toxicity, organs and tissues were harvested at 6 weeks post AAM injections, sectioned, and stained with H&E. Compared to the PBS group, AAM treatment did not induce any noticeable toxicity against all the examined tissues including liver, spleen, kidney, heart, and lung (FIG. 4H).

We also analyzed the potential metabolic labeling of cells in healthy tissues. At 4 days post injections of AAM or PBS, tissues were homogenized and the extracted proteins were incubated with DBCO-biotin prior to western blot analysis. Tissues including brain, liver, spleen, kidney, heart, and lung showed negligible differences between AAM and PBS groups (FIG. 4I). Confocal imaging of tissue sections, after staining with DBCO-Cy5, also showed negligible differences in Cy5 fluorescence intensity between AAM and PBS groups (FIG. 4J). These experiments demonstrated the favorable safety profile of AAM.

### Example 4

### Metabolic Labeling of Red Blood Cell (RBC) Precursor Cells in Bone Marrow and White Blood Cell (WBC) Labeling

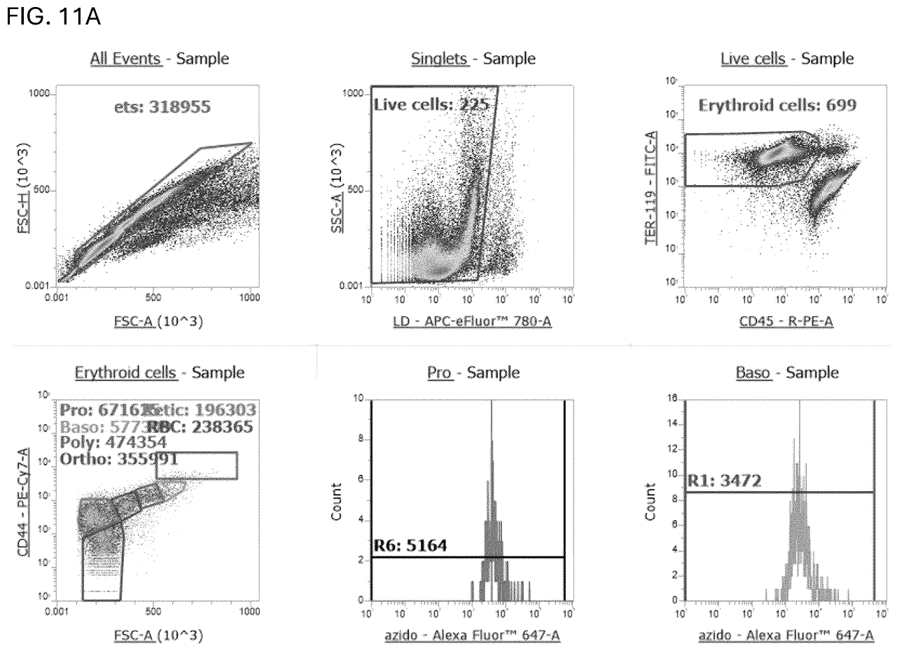

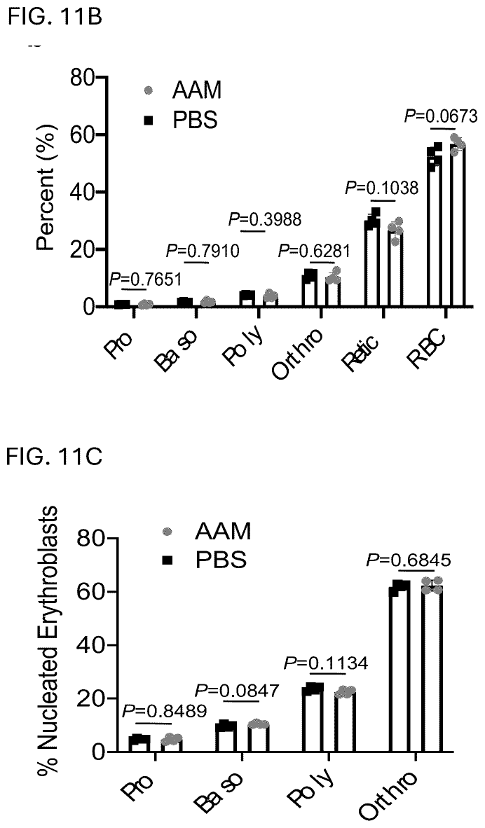

We next studied whether metabolic labeling of RBC precursor cells in the bone marrow contributes to the overall RBC labeling efficiency. Mouse bone marrow cells were harvested at 48 h after AAM injections and stained with DBCO-Cy5 to analyze azido-labeled RBC precursor cells including proerythroblasts, basophilic erythroblasts, polychromatophilic erythroblasts, orthochromatophilic erythroblasts, and reticulocytes (FIG. 11A). Compared to the PBS group, basophilic erythroblasts, polychromatophilic erythroblasts, orthochromatophilic erythroblasts, and reticulocytes in the AAM group showed a higher Cy5 fluorescence intensity (FIG. 3E). The ratio of Cy5 mean fluorescence intensity (AAM/PBS) increased with the stage of erythropoiesis (FIG. 3F). These results demonstrate that systemically administered AAM can metabolically label RBC precursor cells in the bone marrow, which could contribute to the overall RBC labeling efficiency. It is noteworthy that AAM treatment did not alter the percentage and number of erythroid precursor cells (FIGS. 11B-11C). The RBC and WBC counts showed no differences between the AAM and PBS groups (FIG. 4E-4F). Different subtypes of WBCs, including T cells, B cells, and granulocytes, also showed no differences between AAM and PBS groups (FIG. 4G).

### Example 5

### In Vivo Targeting of DBCO-Cargo to Azido-Labeled Red Blood Cells

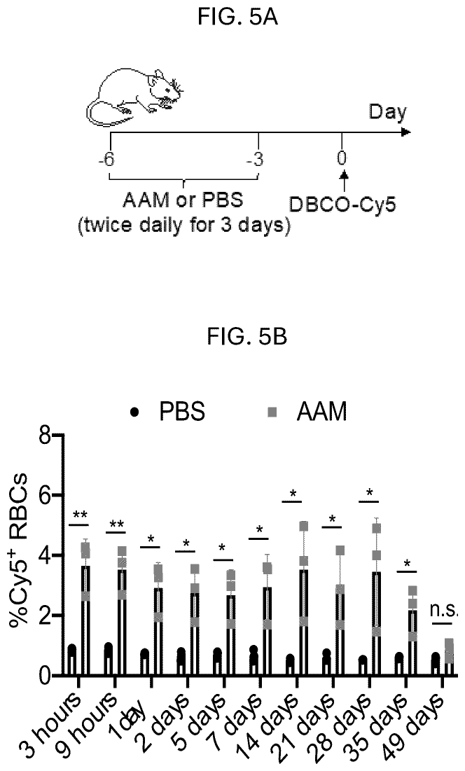

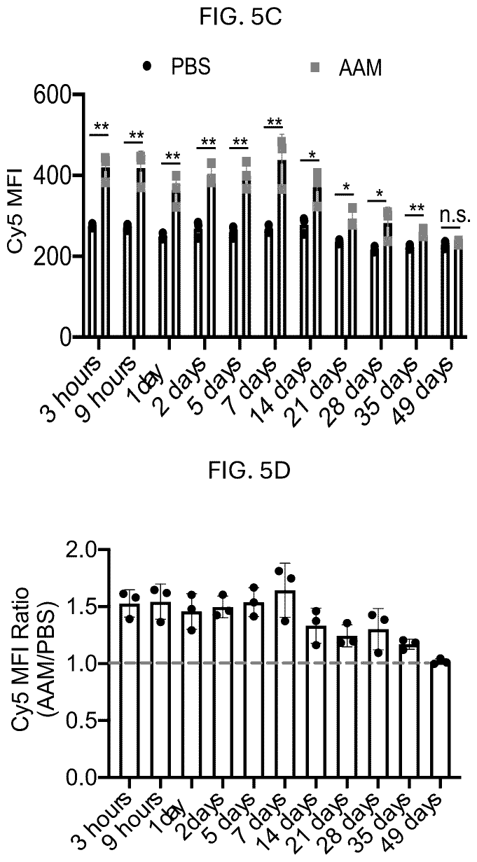

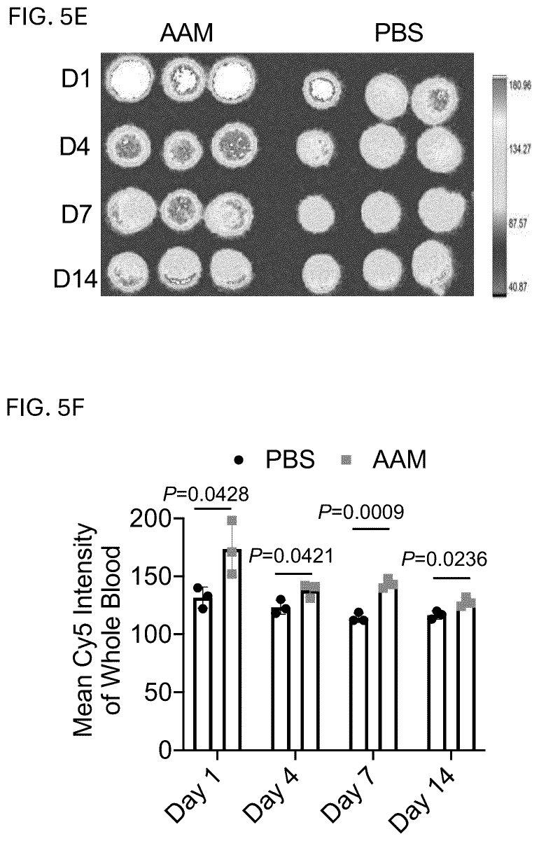

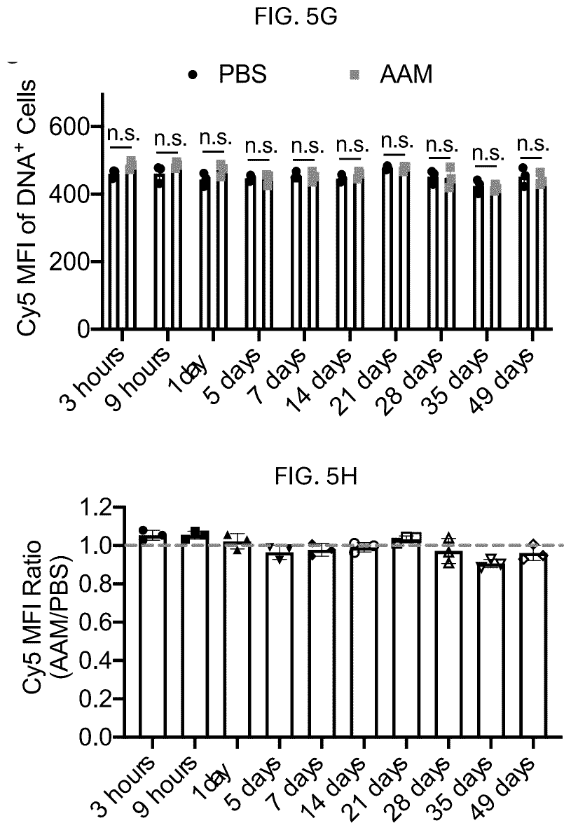

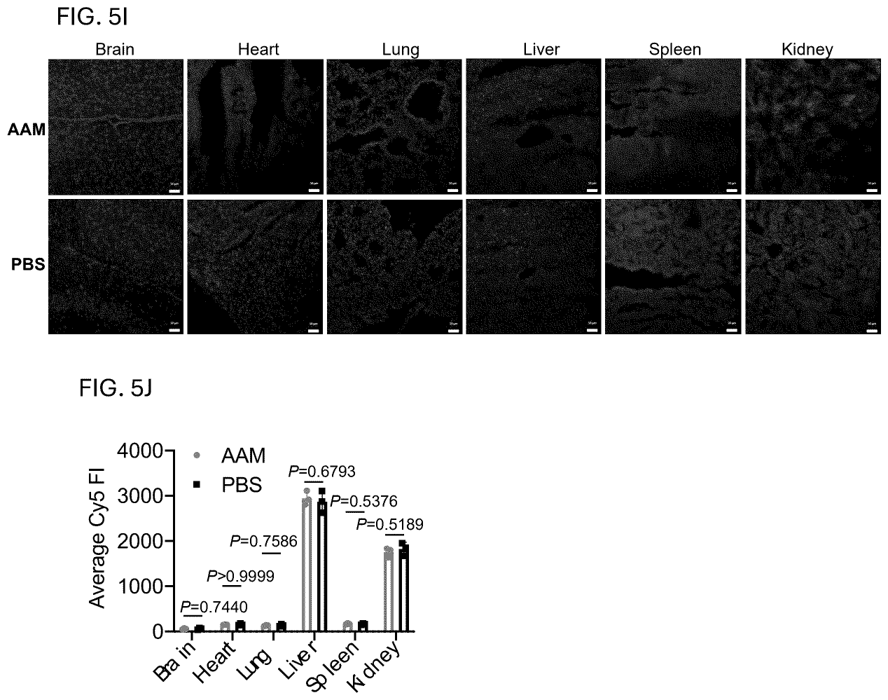

We next studied whether circulating azido-labeled RBCs would enable targeted conjugation of DBCO-molecules via click chemistry in vivo. C57BL/6 mice were intravenously injected with AAM or PBS twice daily for three days, followed by intravenous injection of DBCO-Cy5 at three days post the last injection of AAM (FIG. 5A). At 3 h post injection of DBCO-Cy5, 3.7% of RBCs in AAM-treated mice were Cy5-positive, which was much higher than 0.8% in PBS-treated mice (FIG. 5B). Mean Cy5 fluorescence intensity of RBCs from AAM-treated mice was also significantly higher than control RBCs (FIGS. 5C-5D). At 9 h post injection of DBCO-Cy5, the percentage of Cy5⁺ RBCs and mean Cy5 fluorescence intensity stayed much higher in the AAM-treated mice than in the control mice (FIGS. 5B-5D). After 24 or 48 h when the vast majority of the injected DBCO-Cy5 should have been cleared from the bloodstream, a significantly higher number of Cy5⁺ RBCs were still detected in AAM-treated mice than in control mice (FIGS. 5B-5D).

To monitor the long-term retention of Cy5⁺ RBCs in the bloodstream, we also harvested RBCs at 5, 7, 14, 21, 28, and 35 days, respectively post the injection of DBCO-Cy5, which all showed higher numbers of Cy5⁺ RBCs in AAM-treated mice than in control mice (FIGS. 5B-5D). As expected, blood isolated from AAM-treated mice at days 1, 4, 7, and 14 also showed higher Cy5 fluorescence intensity than blood from PBS-treated mice (FIGS. 5E-5F). Among DNA-containing cells (non-RBCs) in the bloodstream, the accumulation of DBCO-Cy5 showed a negligible difference between AAM and PBS group at all examined times (FIGS. 5E-5H), demonstrating the minimal off-target delivery of DBCO-Cy5 to non-RBC cells. This is consistent with the labeling results that the percentage of azide⁺ cells among DNA-containing blood cells decayed to the baseline at 3 days post injection of AAM (FIGS. 2H-2K).

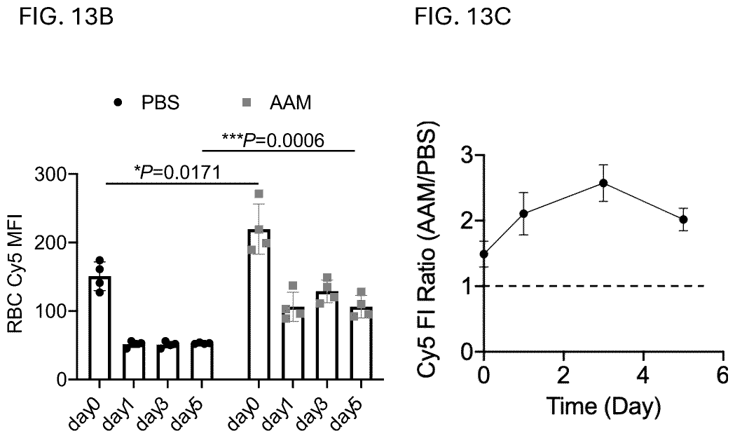

We also examined the potential off-target accumulation of DBCO-Cy5 in healthy tissues. At 24 h post DBCO-Cy5 injection, confocal imaging of tissue sections showed negligible differences in Cy5 fluorescence intensity between AAM and PBS groups (FIGS. 5I-5J). To better understand the membrane retention of conjugated DBCO-Cy5 on RBCs, we also isolated the RBCs at 7 days post injections of AAM, incubated them with DBCO-Cy5, and monitored the membrane retention of Cy5 ex vivo (FIG. 13A). As expected, at 1, 3, and 5 days post DBCO-Cy5 conjugation, RBCs from AAM-treated mice consistently showed much higher Cy5 fluorescence intensity than RBCs from PBS-treated mice (FIGS. 13B-13C), substantiating the excellent membrane retention of Cy5 conjugated onto azido-labeled RBCs. These experiments demonstrated that azido-labeled circulating RBCs can covalently capture DBCO-molecules in vivo and the conjugated molecules can retain on RBC membrane for >5 weeks in mice.

### Example 6

### Long-Term Imaging of Blood Vessels and Tumors

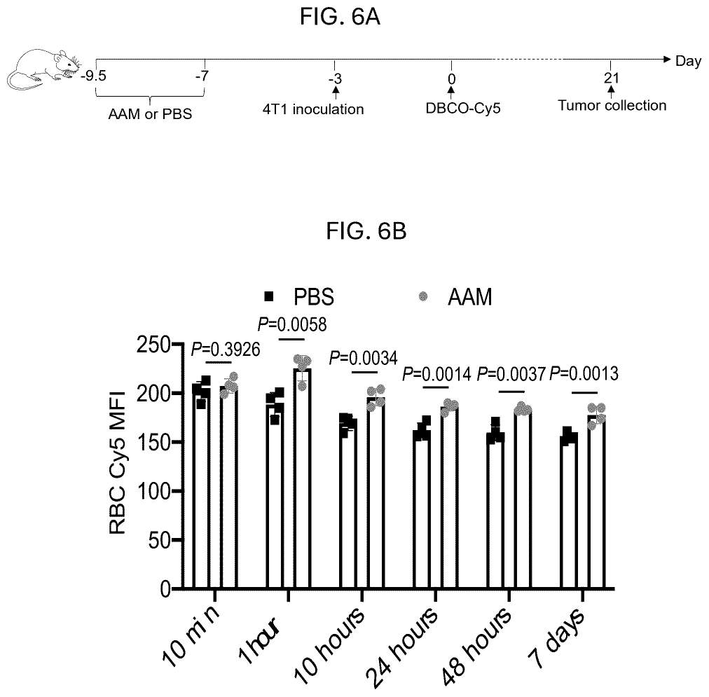

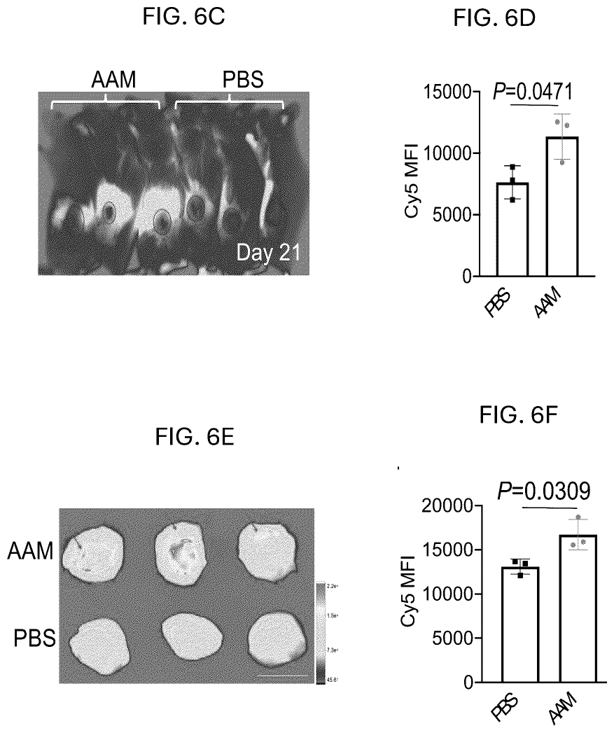

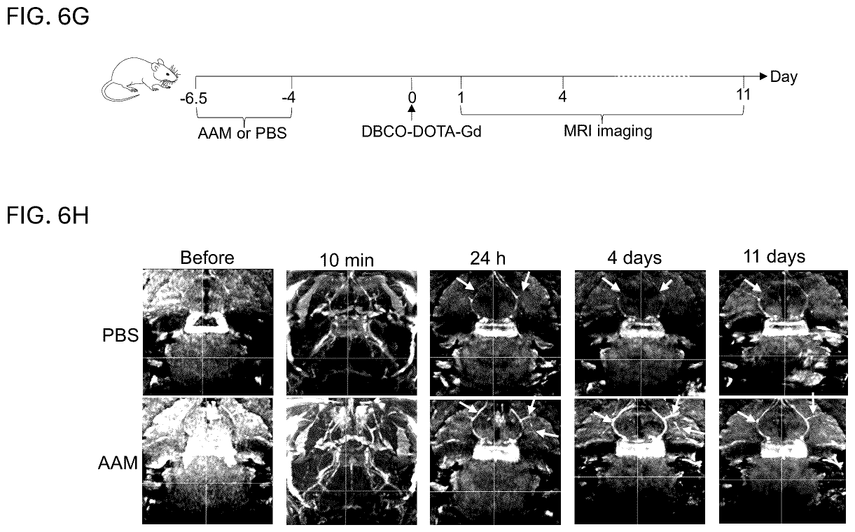

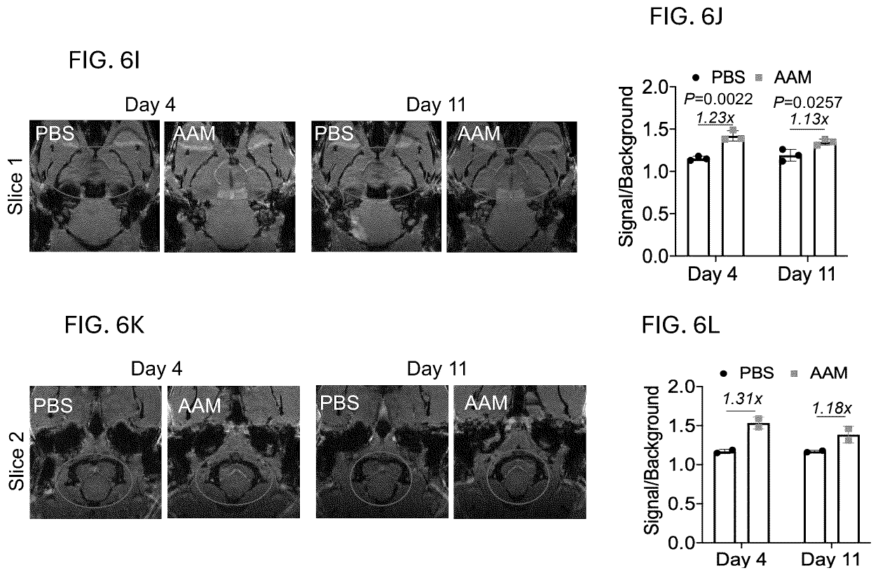

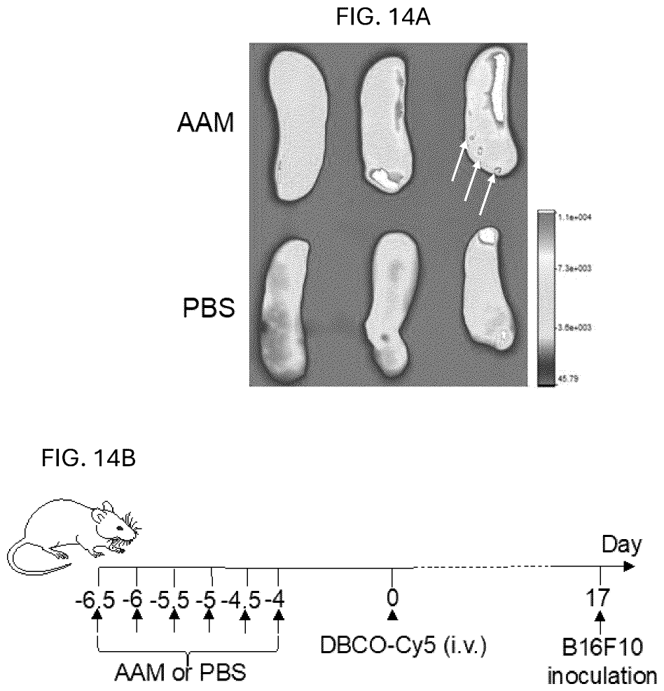

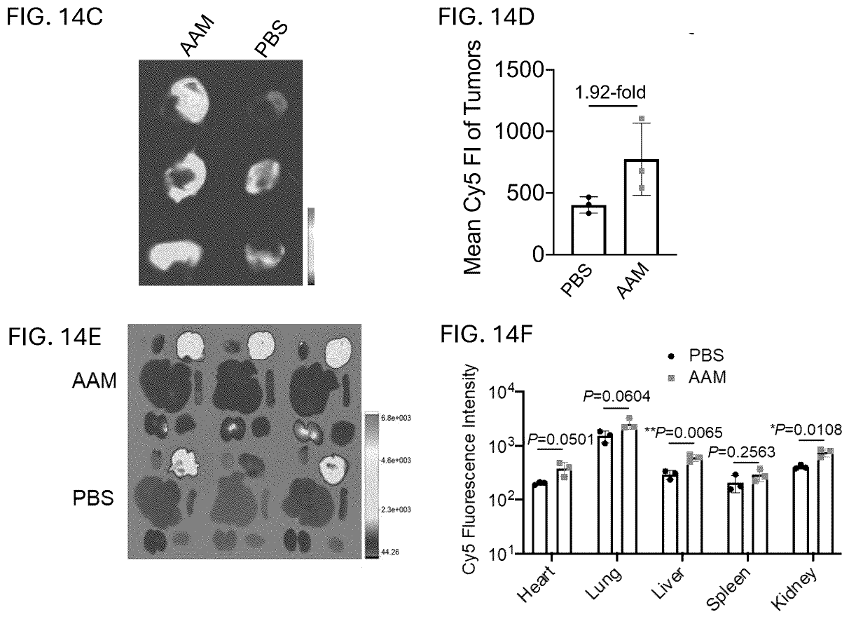

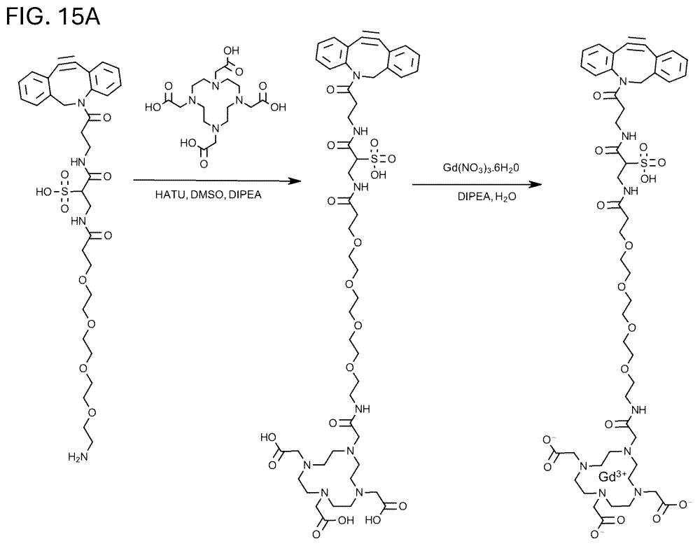

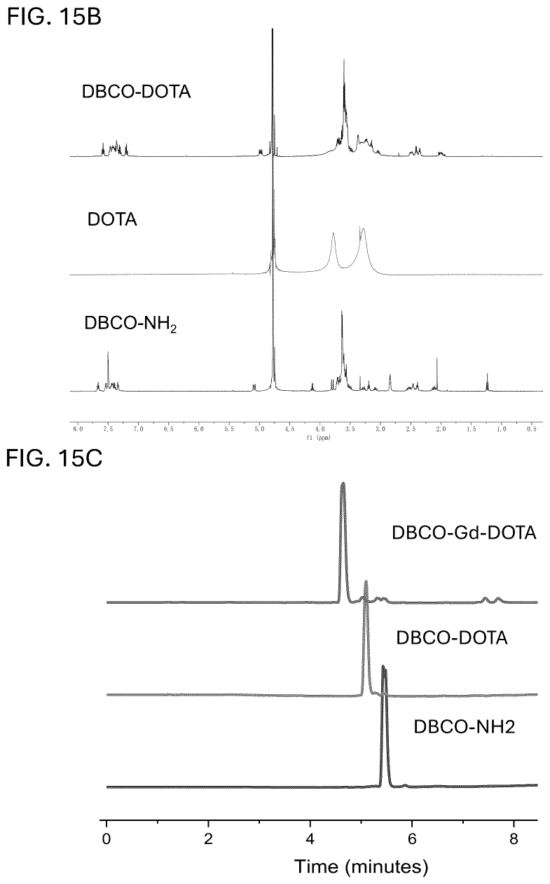

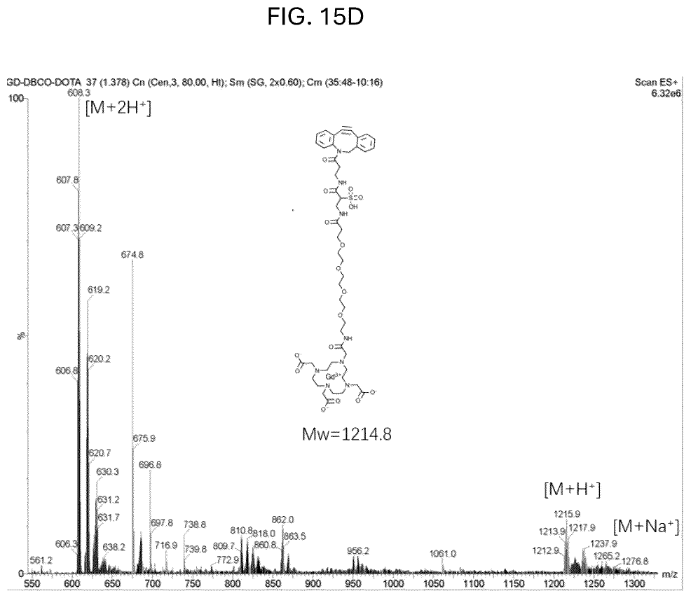

Considering that DBCO-Cy5 can be conjugated to azido-labeled RBCs and be well retained on RBCs in vivo, we next explored its potential for long-term fluorescence imaging of blood vessels and tissues. Balb/c mice were intravenously injected with AAM or PBS twice daily for three days, followed by the subcutaneous inoculation of 4T1 tumor cells and intraperitoneal injection of DBCO-Cy5 at 4 and 7 days, respectively post injections of AAM (FIG. 6A). Starting from 1 h post injection of DBCO-Cy5, a higher Cy5 fluorescence intensity was detected in RBCs from AAM-treated mice than RBCs from PBS-treated mice (FIG. 6B). Starting from 1 h post injection of DBCO-Cy5, a higher Cy5 fluorescence intensity was detected in RBCs from AAM-treated mice than RBCs from PBS-treated mice (FIG. 6B). IVIS imaging also showed a higher Cy5 fluorescence signal (˜1.5 fold) of 4T1 tumors in AAM-treated mice than in PBS-treated mice at 21 days post DBCO-Cy5 injection (FIGS. 6C-6D). Ex vivo imaging of 4T1 tumors harvested at 24 days post tumor inoculation confirmed a higher Cy5 fluorescence signal in the AAM group (FIGS. 6E-6F). Indeed, blood vessels in 4T1 tumors of AAM-treated mice could be clearly visualized (FIG. 6E). Similarly, the blood vessels in other tissues, including the terminal arterioles structures in the spleen, could be visualized (FIG. 14A).

In another setting, C57BL/6 mice were intravenously injected with AAM or PBS twice daily for three days, followed by intravenous injection of DBCO-Cy5 on day 0 and B16F10 tumor inoculation on day 17 (FIG. 14B). On day 24, i.e., 7 days post the subcutaneous inoculation of B16F10 cells, B16F10 tumors from AAM-treated mice showed a 1.92-fold Cy5 fluorescence intensity in comparison with tumors from PBS-treated mice (FIGS. 14C-14D), presumably due to the presence of Cy5-conjugated RBCs within the tumor vasculatures. Consistently, we observed a higher Cy5 fluorescence signal in the liver, kidney, and other organs of AAM-treated mice than those of PBS-treated mice (FIGS. 14E-14F).

As cargos can be conjugated to circulating RBCs and retain on RBCs for weeks via the RBC labeling and targeting technology, we next investigated whether MRI contrast agents (e.g., Gd) can be tagged onto RBCs in vivo for long-term MRI, using contrast-enhanced (CE) imaging of brain blood vessels as an example. We first synthesized DBCO-DOTA-Gd, by reacting DBCO-NH₂ with DOTA-COOH to yield DBCO-DOTA and furthering complexing DBCO-DOTA and Gd³⁺ (FIG. 13A). The chemical structure of DBCO-DOTA and DBCO-DOTA-Gd was characterized by ¹H NMR spectroscopy (FIG. 13B), HPLC (FIG. 13C), and mass spectrometry (FIG. 13D). For the in vivo MRI study, C57BL/6 mice were intravenously injected with AAM or PBS twice daily for three days, followed by intravenous injection of DBCO-DOTA-Gd after 4 days (FIG. 6G). Before the injection of DBCO-DOTA-Gd, the blood vessels were barely visible in a CE T1-weighted MR angiography scan (CE-MRA), with or without maximum intensity projection (MIP) (FIG. 6H). At 10 min post the injection of DBCO-DOTA-Gd, blood vessels were clearly visible in both AAM- and PBS-treated mice using the same CE-MRA sequence (FIG. 6H). At 24 h when free DBCO-DOTA-Gd was largely cleared, while MIP could visualize the main blood vessels in both AAM and PBS group, only the AAM group showed visible branched vessels (FIG. 6H), showing excellent retention of DBCO-DOTA-Gd on the azido-labeled RBCs. Without RBC labeling, Gd would need to be injected again for CE-MRA scans, as demonstrated in the PBS group. Strikingly, this signal enhancement was still detected even at 4 or 11 days post injection of DBCO-DOTA-Gd for the AAM group (FIG. 6H). To further compare the signals, we analyzed two specific coronal views of the raw MR images for both day 4 and day 11. Compared to PBS-treated mice, AAM-treated mice showed enhanced MR signals in several blood vessels on both day 4 and day 11 (FIGS. 6I, 6K). Quantification of the signals indicated a 1.23-fold enhancement for AAM group on day 4 and a 1.13-fold enhancement on day 11 (FIG. 6J), which was consistent in different coronal views (FIGS. 6J, 6L). These experiments demonstrated the feasibility of conjugating Gd to circulating RBCs for long-term MRI of blood vessels.

### Example 7

### In Vivo Conjugation of Drugs to Red Blood Cells for Improved Pharmacokinetics and Efficacy

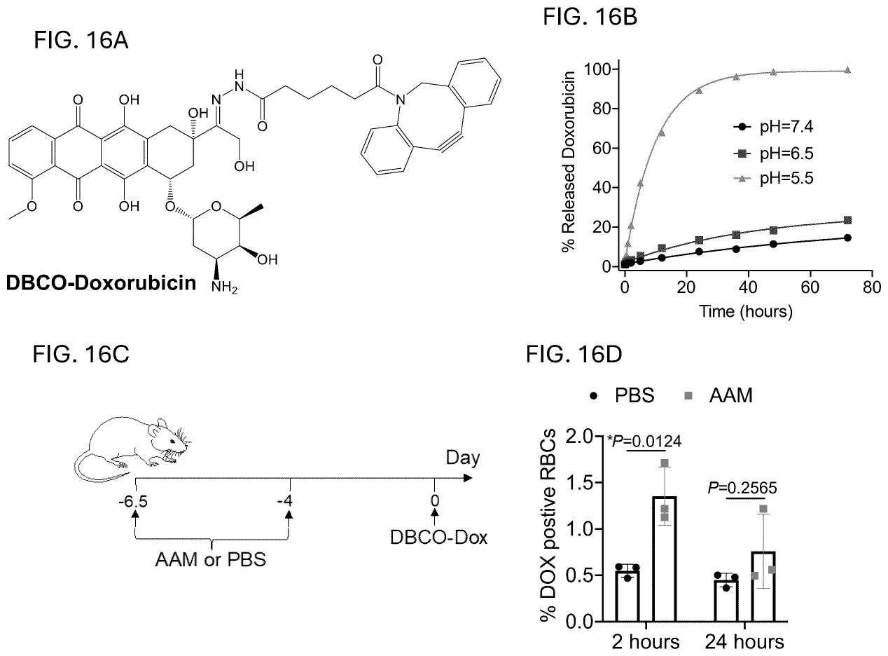

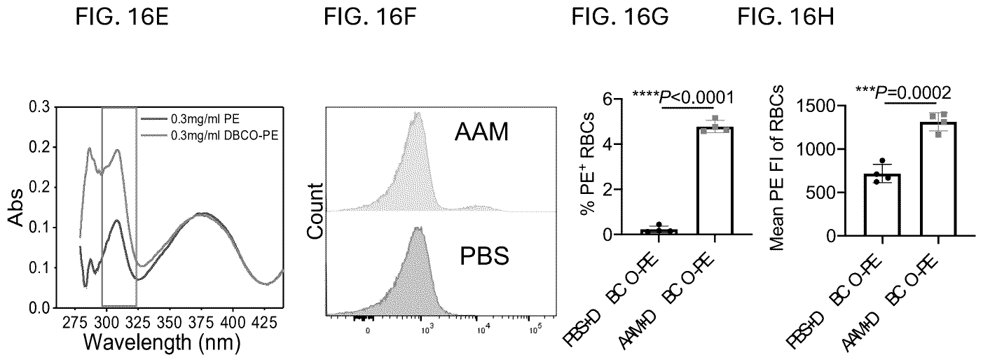

In addition to imaging agents, the RBC labeling and targeting approach could be utilized to improve the pharmacokinetics of therapeutics. The feasibility of conjugating small-molecule doxorubicin and macromolecular phycoerythrin (PE) onto azido-labeled RBCs was first tested. DBCO-doxorubicin with a pH-labile hydrazone linkage was synthesized (FIGS. 16A-16B). Following intravenous injection of AAM or PBS into C57BL/6 mice twice daily for three days, DBCO-doxorubicin was intravenously administered (FIG. 16C). At 2 h post the injection of DBCO-doxorubicin, a higher fraction of doxorubicin-containing RBCs was detected in AAM-treated mice than in control mice (FIG. 16D). Similarly, PE, upon DBCO functionalization (FIG. 16E), can conjugate to azido-labeled RBCs via click chemistry (FIGS. 16F-16H).

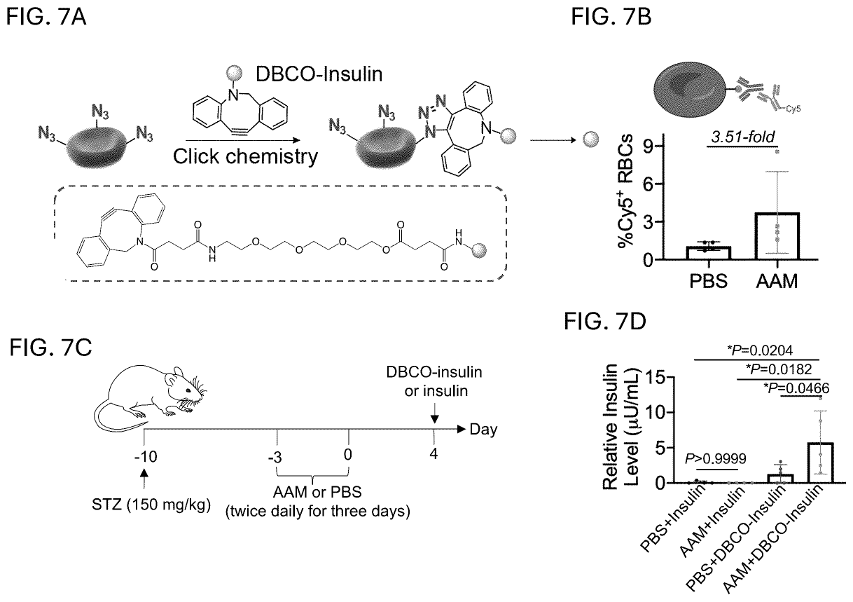

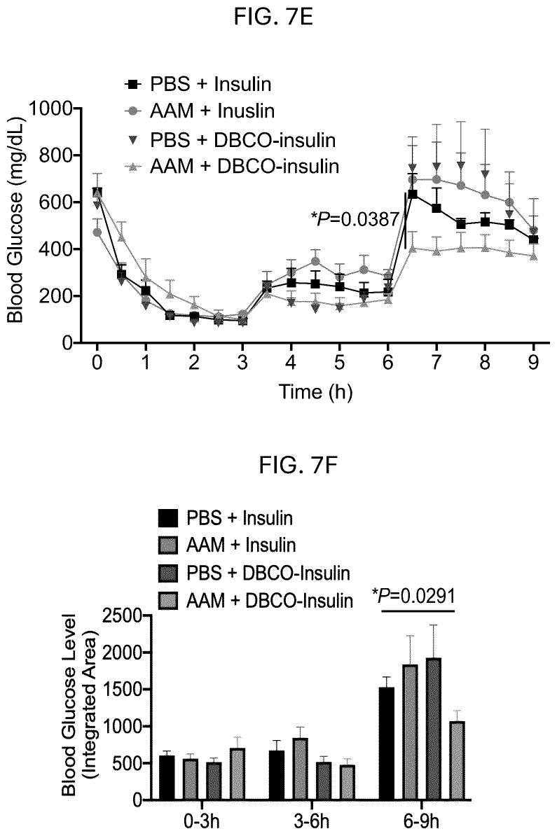

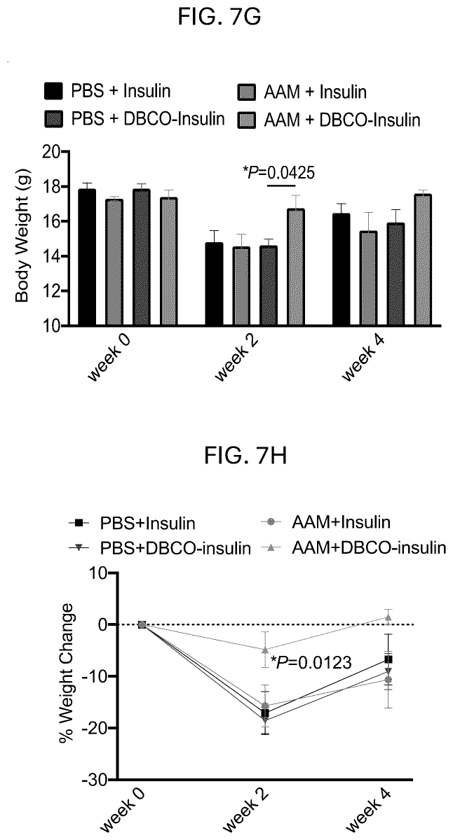

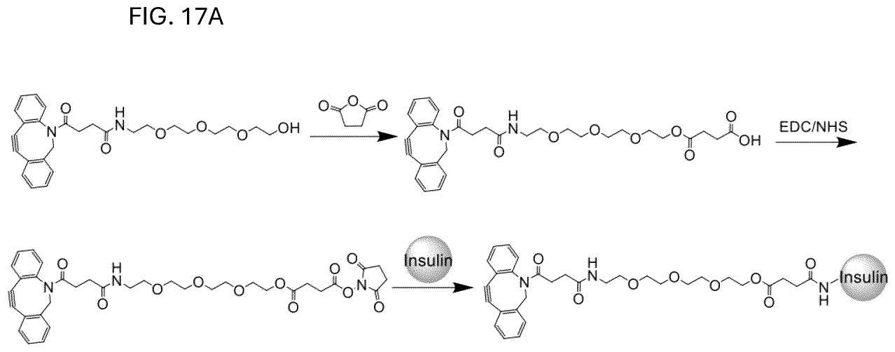

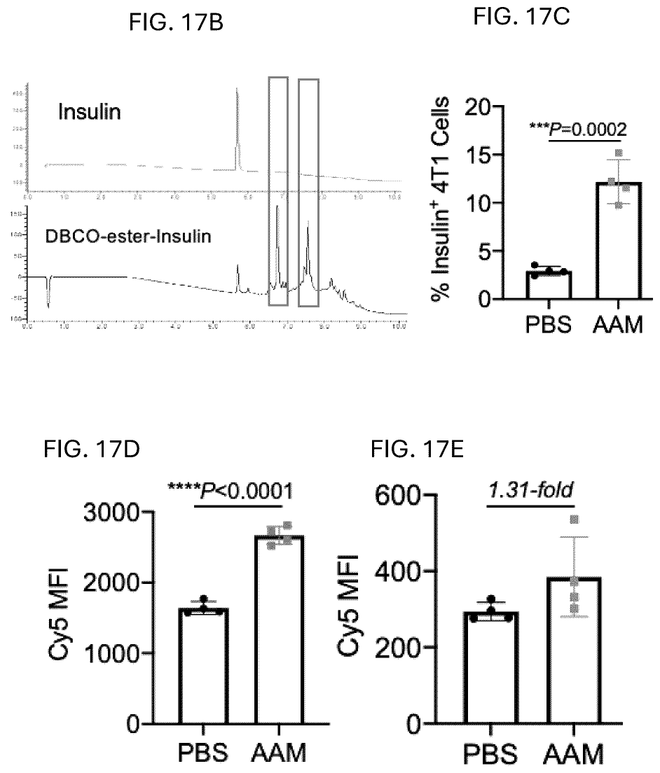

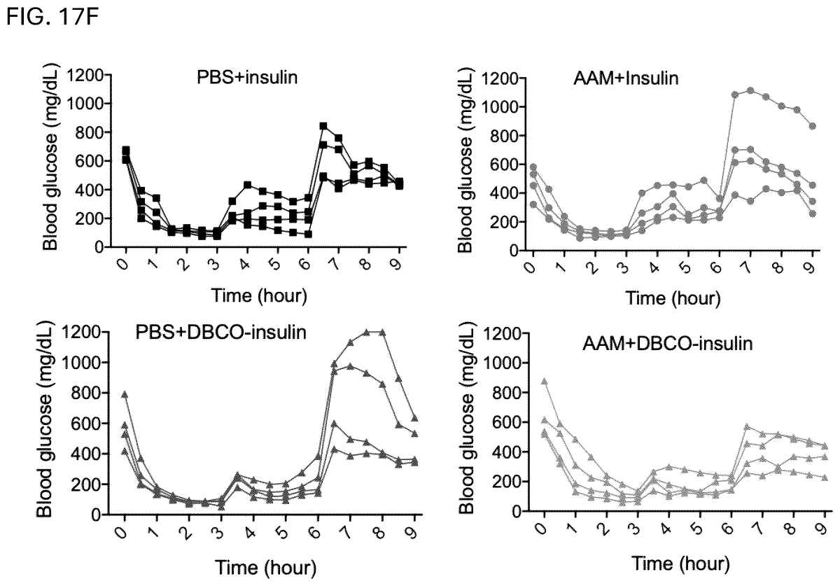

After demonstrating the feasibility of conjugating different cargos to RBCs, we next explored whether DBCO-insulin can be conjugated to circulating RBCs for improved pharmacokinetics and therapeutic efficacy. DBCO-insulin with a hydrolysable ester linkage was synthesized and characterized (FIG. 7A, FIGS. 17A-17B). By incubating AAM- or PBS-treated 4T1 cells with DBCO-insulin and detecting the cell-surface insulin using rabbit anti-insulin and Cy5-conjugated goat anti-rabbit secondary antibody, a higher Cy5 fluorescence signal was detected in AAM-treated 4T1 cells than in control cells (FIGS. 17C-17D), demonstrating the successful conjugation of DBCO-insulin. DBCO-insulin was also able to conjugate to azido-labeled RBCs that were isolated from AAM-treated mice, as evidenced by antibody staining (FIG. 7B, FIG. 17E). In vivo, following the intravenous injection of AAM or PBS into streptozotocin (STZ)-pretreated mice, DBCO-insulin or unmodified insulin was intraperitoneally injected. At 8 h post the injection, a significantly higher level of insulin was detected in the plasma of mice injected with AAM and DBCO-insulin than the control mice (FIGS. 7C-7D), demonstrating the successful conjugation of DBCO-insulin to azido-labeled RBCs in vivo and subsequent release of insulin from RBCs. In the subsequent glucose tolerance test, AAM-mediated RBC labeling combined with DBCO-insulin improved the control of blood glucose levels, in comparison with the non-targeting groups (FIGS. 7E-7F, FIG. 17F). Consistent with the glucose control result, diabetic mice treated with AAM and DBCO-insulin showed the fastest recovery in body weight than other groups (FIGS. 7G-7H). These experiments demonstrated the promise of the RBC labeling and targeting technology to enhance the pharmacokinetics and therapeutic efficacy of drugs.

In view of the many possible implementations to which the principles of the disclosure may be applied, it should be recognized that the illustrated implementations are only examples and should not be taken as limiting the scope of the invention. Rather, the scope of the invention is defined by the following claims. We therefore claim as our invention all that comes within the scope and spirit of these claims.

We claim:

1. A method for treating or inhibiting a disease, comprising:
administering to a subject a composition comprising an azido-modified sugar moiety, thereby generating red blood cells comprising one or more azido-labeled surface proteins; and
administering to the subject a composition comprising a cargo for treating or inhibiting the disease, wherein the cargo is capable of covalently binding to the one or more azido-labeled surface proteins or lipids.

2. The method of claim 1, wherein the composition comprising the cargo for treating or inhibiting the disease is administered to the subject after the composition comprising the azido-modified sugar moiety.

3. The method of claim 2, wherein the composition comprising the cargo for treating or inhibiting the disease is administered to the subject at least about 3 days after the composition comprising the azido-modified sugar moiety.

4. The method of claim 1, wherein the composition comprising an azido-modified sugar moiety, the composition comprising a cargo for treating or inhibiting the disease, or both is administered to the subject intravenously.

5. The method of claim 1, wherein the azido-labeled sugar moiety is tetra-acetylated N-azidoacetyl-D-mannosamine (Ac₄ManNAz), N-azidoacetyl-D-mannosamine (ManNAz), tetra-acetylated N-azidoacetyl-D-galactosamine (Ac₄GalNAz), N-azidoacetyl-D-galactosamine (GalNAz), tetra-acetylated N-azidoacetyl-D-glucosamine (Ac₄GlcNAz), N-azidoacetyl-D-glucosamine (GlcNAz), or N-Acetyl-9-azido-9-deoxy-neuraminic acid (9-AzNeu5Ac).

6. The method of claim 1, wherein the cargo capable of covalently binding to the one or more azido-labeled surface proteins or lipids is capable of covalent binding by click chemistry.

7. The method of claim 6, wherein the click chemistry is azide-alkyne click chemistry.

8. The method of claim 1, wherein the cargo capable of covalently binding to the one or more azido-labeled surface proteins or lipids comprises dibenzocyclooctyne.

9. The method of claim 1, wherein:
the cargo is a cancer therapeutic compound and the disease is cancer; or
the cargo is insulin and the disease is diabetes; or
the cargo is an antigen and the disease is an infection; or
the cargo is an autoantigen and the disease is an autoimmune disorder; or
the cargo is an antibiotic and the disease is a bacterial infection; or
the cargo is an antibiotic and the disease is a viral infection.

10. A method of in vivo imaging, comprising:
administering to a subject a composition comprising an azido-modified sugar moiety, thereby generating red blood cells comprising one or more azido-labeled surface proteins;
administering to the subject a composition comprising an imaging agent, wherein the imaging agent is capable of covalently binding to the one or more azido-labeled surface proteins or lipids; and
performing an imaging analysis of the subject one or more times.

11. The method of claim 10, wherein the imaging agent is a contrast agent, a fluorescent dye, or a bioluminescent substrate.

12. The method of claim 10, wherein the imaging analysis is magnetic resonance imaging.

13. A method for treating or inhibiting a disease, comprising:
contacting a preparation of red blood cells obtained from a subject with the disease or disorder with a composition comprising an azido-modified sugar for a sufficient period of time for the red blood cells to express one or more azido-labeled surface proteins or lipids;
contacting the red blood cells expressing one or more azido-labeled surface proteins or lipids with a composition comprising a cargo for treating or inhibiting the disease, wherein the cargo is capable of covalently binding to the one or more azido-labeled surface proteins or lipids, thereby producing a population of modified red blood cells; and
administering the population of modified red blood cells to the subject.

14. The method of claim 13, wherein the azido-labeled sugar moiety is tetra-acetylated N-azidoacetyl-D-mannosamine (Ac₄ManNAz), N-azidoacetyl-D-mannosamine (ManNAz), tetra-acetylated N-azidoacetyl-D-galactosamine (Ac₄GalNAz), N-azidoacetyl-D-galactosamine (GalNAz), tetra-acetylated N-azidoacetyl-D-glucosamine (Ac₄GlcNAz), N-azidoacetyl-D-glucosamine (GlcNAz), or N-Acetyl-9-azido-9-deoxy-neuraminic acid (9-AzNeu5Ac).

15. The method of claim 13 wherein the population of red blood cells from the subject is contacted with the azido-labeled sugar moiety for about 1-120 hours.

16. The method of claim 13, wherein the cargo capable of covalently binding to the one or more azido-labeled surface proteins or lipids is capable of covalent binding by click chemistry.

17. The method of claim 16, wherein the click chemistry is azide-alkyne click chemistry.

18. The method of claim 13, wherein the cargo capable of covalently binding to the one or more azido-labeled surface proteins or lipids comprises dibenzocyclooctyne.

19. A modified red blood cell comprising one or more surface proteins or lipids covalently linked to a cargo by click chemistry.

20. The modified red blood cell of claim 19, wherein the click chemistry is azide-alkyne click chemistry, tetrazine-norbornene click chemistry, tetrazine-cyclooctene click chemistry, or maleimide-thiol click chemistry.

21. The modified red blood cell of claim 19, wherein the cargo is a cancer therapeutic compound, insulin, an antigen, an autoantigen, an antibiotic, an antiviral, an antibody, or an imaging agent.

22. The modified red blood cell of claim 19, wherein the click chemistry comprises an azido-labeled sugar moiety.

23. The modified red blood cell of claim 22, wherein the azido-labeled sugar moiety is tetra-acetylated N-azidoacetyl-D-mannosamine (Ac₄ManNAz), N-azidoacetyl-D-mannosamine (ManNAz), tetra-acetylated N-azidoacetyl-D-galactosamine (Ac₄GalNAz), N-azidoacetyl-D-galactosamine (GalNAz), tetra-acetylated N-azidoacetyl-D-glucosamine (Ac₄GlcNAz), N-azidoacetyl-D-glucosamine (GlcNAz), or N-Acetyl-9-azido-9-deoxy-neuraminic acid (9-AzNeu5Ac).

24. A composition comprising the modified red blood cell of claim 19 and a pharmaceutically acceptable carrier.
